SEQUENCE LISTING (11 sequences)

SEQ ID NO: 1 - RNA, 22 nt, Homo sapiens
guguuugguaauacacgacgau

SEQ ID NO: 2 - RNA, 21 nt, Homo sapiens
cugcugcugcugcugcugcug

SEQ ID NO: 3 - RNA, 21 nt, Homo sapiens
gccgagcccaccgccgcugcc

SEQ ID NO: 4 - RNA, 21 nt, Homo sapiens
cggcggcggcggcggcggcgg

SEQ ID NO: 5 - RNA, 22 nt, Homo sapiens
cgagccggggccggggucgggg

SEQ ID NO: 6 - RNA, 22 nt, Homo sapiens
ccccggccccggccccggcccc

SEQ ID NO: 7 - RNA, 22 nt, Homo sapiens
gugaaucguccaacauaauaua

SEQ ID NO: 8 - RNA, 17 nt, Homo sapiens
auuguauuguauuguau

SEQ ID NO: 9 - RNA, 22 nt, Homo sapiens
cgccagggcgccgcggggcgga

SEQ ID NO: 10 - RNA, 24 nt, Homo sapiens
cgcgccccgccccgcgccccgccc

SEQ ID NO: 11 - DNA, 40 bp, Artificial Sequence
  Control olio for non-specific binding
cccgccccgccgcgcccccgccccgccgcgcccccgcccc

(19) United States
(12) Patent Application Publication
(10) Pub. No.: US 2016/0340732 A1
(43) Pub. Date: Nov. 24, 2016

(54) COMPOSITIONS AND METHODS FOR DIAGNOSING AND TREATING NUCLEOTIDE REPEAT DISORDERS

(71) Applicant: University of Virginia Patent Foundation, Charlottesville, VA (US)

(72) Inventors: Erin P. FOFF, Charlottesville, VA (US); Benjamin W. PUROW, Charlottesville, VA (US)

(21) Appl. No.: 15/156,626

(22) Filed: May 17, 2016

Related U.S. Application Data
(60) Provisional application No. 62/163,051, filed on May 18, 2015.

(51) Int. Cl. C12Q 1/68; A61K 31/7088; A61K 31/167; A61K 31/16; A61K 31/19

(52) U.S. Cl. CPC C12Q 1/6883; A61K 31/16; A61K 31/19; A61K 31/167; A61K 31/7088; C12Q 2600/106; C12Q 2600/178; C12N 2740/15042

(57) ABSTRACT

The present application discloses compositions and methods useful for diagnosing and treating nucleotide repeat disorders. An in silico analysis indicates a striking and specific miR-NRD homology. Validated miR target prediction software was used to assess each NRD nucleotide repeat expansion for potential miR homology. The striking degree of matched homology and the one-to-one expansion-to-miR ratio strongly support their relevance to the corresponding NRD and serve as a basis for therapeutic treatments. It is also disclosed herein that NETO is involved in mediating C9-repeat glutamate excitotoxicity, thus representing a novel therapeutic target.

### CROSS REFERENCE TO RELATED APPLICATIONS

This application claims priority to U.S. Provisional Patent Application No. 62/163,051 filed on May 18, 2016, the disclosure of which is hereby expressly incorporated by reference in its entirety.

### BACKGROUND

Expansions of small DNA motifs cause nucleotide repeat disorders (NRDs), which are some of the most debilitating diseases we treat. These include Huntington's disease (HD), myotonic dystrophy (DM1), and inherited forms of amyotrophic lateral sclerosis (ALS) and frontotemporal dementia (FTD). The DNA expansions may disrupt many aspects of normal cell machinery, but a unifying mechanism across the NRDs has not been found. Rather, investigators have proposed diverse mechanisms of pathogenesis, from aberrant proteins/peptides causing loss or gain of protein function to toxic RNA foci that modulate RNA binding proteins to creation of aberrant microRNAs (miRs) from the expanded repeat locus.

### SUMMARY

The present application discloses compositions and methods useful for diagnosing and treating nucleotide repeat disorders. More particularly, in one embodiment the present application is directed to diagnosing and treating Huntington's disease (HD) and familial amyotrophic lateral sclerosis with frontotemporal dementia (C9ORF72 ALS/FTD, referred to hereafter as C9+ ALS). An in silico analysis indicates a striking and specific miR-NRD homology among various diseases associated with nucleotide repeat disorders.

It is also disclosed herein that NETO is involved in mediating C9-repeat glutamate excitotoxicity, thus representing a novel therapeutic target. Therefore, the present invention provides compositions and methods for targeting and regulating NETO pathways and C9-repeat pathways and activity.

There has been a lack of a unifying mechanism for the Nucleotide Repeat Disorders. Without wishing to be bound by any particular theory, it is hypothesized herein that all nucleotide expansion motifs act as sponges for specific miRs and thus share a common and novel mechanism for disease pathogenicity. Additionally, the present disclosure encompasses a completely novel mechanism for NRD pathogenesis, and a radical new approach to treatment based on that mechanism. Furthermore, it suggests a unifying mechanism for 25 diseases previously treated as unique from one another, and enables targeting an upstream step in pathogenesis.

In accordance with one embodiment a method of treating a nucleotide repeat disorder in a patient is provided. In one embodiment the method comprises detecting the presence of one or more nucleotide repeats, that are associated with a nucleotide repeat disorder (NRD), in a biological sample recovered from said patient, and treating those patients having a nucleotide repeat disorder by administering an effective amount of a therapeutic to the diagnosed patient. In one embodiment the biological sample is screened for the presence of a nucleotide sequence selected from the group consisting of SEQ ID NO: 2, 4, 6, 8 and 10. In one embodiment the administered therapeutic enhances the activity of an miRNA selected from the group consisting of SEQ ID NO: 1, 3, 5, 7, and 9. In one embodiment the method comprises identifying patients having a nucleotide repeat disorder and treating the identified nucleotide repeat disorder (NRD) patients with an HDAC inhibitor selected from the group consisting of is trichostatin A, valproate and vorinostat.

In accordance with one embodiment a method of treating patient having ALS is provided. The method comprises screening a biological sample recovered from a patient for the presence of a C9 expansion (e.g., SEQ ID NO: 6, or its complement thereof). Those patients that comprise the C9 expansion sequence in their genome will be treated with a therapeutic regiment that
a) enhances activity of miR-762,
b) decreasing activity of NETO1, or
c) both a) and b). In accordance with one embodiment, those patients that comprise the C9 expansion sequence in their genome will be treated by the administration of a composition comprising an effective amount of an HDAC inhibitor.

Various aspects and embodiments of the invention are described in further detail below.

### BRIEF DESCRIPTION OF THE DRAWINGS

FIG. 1 is an illustrative view of an unaffected stretch of DNA and an affected stretch of DNA having a nucleotide repeat expansion.

FIG. 2 is a diagrammatic view of a luciferase based assay used to test the activity of miRNAs showing that when a specific miR binds its match in 3′UTR of the Luc reporter, expression of luciferase is suppressed. In addition, if the miR is sponged (i.e., miR activity effectively reduced) the luciferase expression increases.

FIG. 3 shows normalized miR-15 levels of three different constructs using the luciferase assay.

FIG. 4 shows normalized miR-195 levels for three different constructs using the luciferase assay.

FIG. 5 shows normalized miR-1181 levels for four different constructs using the luciferase assay.

FIG. 6 shows miR-762 activity in the presence of C9+ repeats in the luciferase based assay.

FIG. 7 shows miR-762 levels in mouse C2C12 cells treated with TSA (50 nM) or DMSO for 48 hours.

FIG. 8 shows miR-762 levels in mouse C2C12 cells treated with valproate (1 mM) or H₂O for 48 hours.

FIG. 9 is a graph of B-lymphocytes from three C9(+) and three C9(−) patients that were transfected with a miR-762 reporter plasmid, and further showing increased luciferase activity in the C9(+) cells indicating decreased miR-762 activity.

FIG. 10 shows NETO1 mRNA is increased in C9+B-lymphocytes compared to a population control and C9 (−) lymphocytes.

FIG. 11 shows NETO1 protein levels in C9(−) and C9(+) cells.

FIG. 12 shows a comparison of NETO1 mRNA from C9(−) ALS patients and C9(+) ALS patients.

FIG. 13 shows mRNA levels of NETO1 from the frontal cortex of Sporadic ALS and C9(+) ALS patients.

FIG. 14 shows NETO1 RNA level fold change from transfecting a (C₄G₂)₁₂ construct.

FIG. 15 shows NETO1 mRNA levels in astrocytes transfected with constructs having a miR-762 sequence, a scrambled miR-762 sequence, or an miR-762 antisense inhibitor.

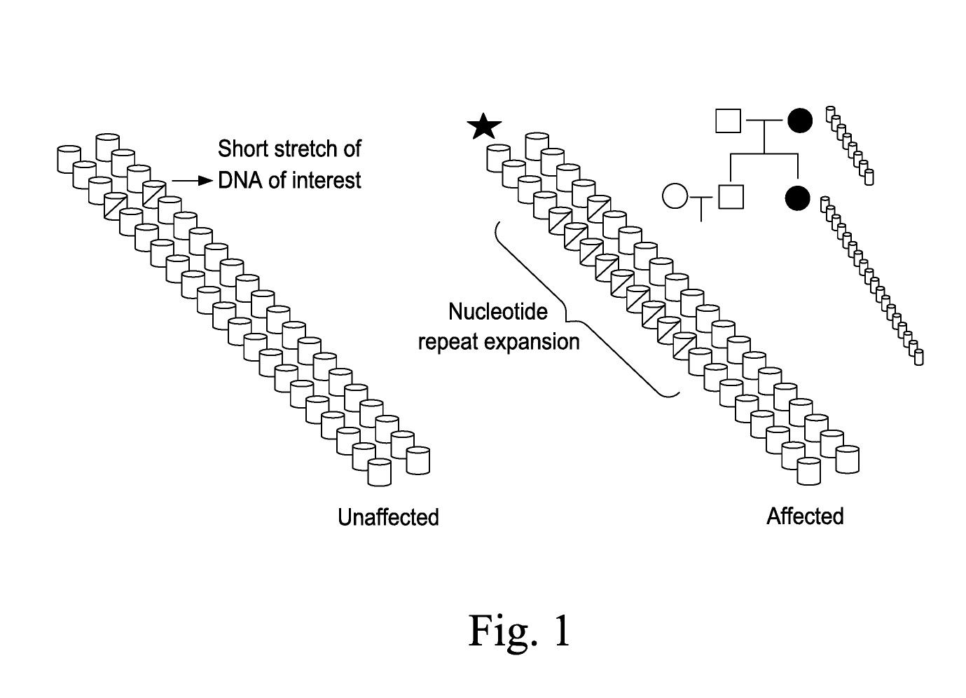

### DETAILED DESCRIPTION

### Abbreviations and Acronyms

Alzheimer's Disease (AD)

brain-derived neurotrophic factor (BDNF)

Fragile X Tremor-Associated Syndrome (FXTAS)

Frontotemporal lobar degeneration/dementia (FTLD or FTD)

nucleotide repeat disorders (NRDs)

Huntington's disease (HD)

myotonic dystrophy (DM1)

NETO1 (Neuropilin tolloid-like1)

amyotrophic lateral sclerosis (ALS)

Repeat-Associate Non-ATG (RAN)

nucleolin (NCL)

microRNA (miR or miRNA)

neural stem cell (NSC)

nucleotide repeat expansion (NRE)

### DEFINITIONS

In describing and claiming the invention, the following terminology will be used in accordance with the definitions set forth below.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

The term “alterations in peptide structure” as used herein refers to changes including, but not limited to, changes in sequence, and post-translational modification.

As used herein, “alleviating a disease or disorder symptom,” means reducing the severity of the symptom or the frequency with which such a symptom is experienced by a patient, or both.

As used herein, amino acids are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

Amino acids have the following general structure:

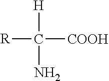

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antagomir” refers to a small RNA or DNA (or chimeric) molecule to antagonize endogenous small RNA regulators like microRNA (miRNA). These antagonists bear complementary nucleotide sequences for the most part, which means that antagomirs should hybridize to the mature microRNA (miRNA). They prevent other molecules from binding to a desired site on an mRNA molecule and are used to silence endogenous microRNA (miR). Antagomirs are therefore designed to block biological activity of these post-transcriptional molecular switches Like the preferred target ligands (microRNA, miRNA), antagomirs have to cross membranes to enter a cell. Antagomirs also known as anti-miRs or blockmirs.

An “antagonist” is a composition of matter which when administered to a mammal such as a human, inhibits a biological activity attributable to the level or presence of a compound or molecule of interest in the mammal.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

As used herein, the term “attach”, or “attachment”, or “attached”, or “attaching”, used herein interchangeably with “bind”, or “binding” or “binds' or “bound” refers to any physical relationship between molecules that results in forming a stable complex, such as a physical relationship between a ligand, such as a peptide or small molecule, with a “binding partner” or “receptor molecule.” The relationship may be mediated by physicochemical interactions including, but not limited to, a selective noncovalent association, ionic attraction, hydrogen bonding, covalent bonding, Van der Waals forces or hydrophobic attraction.

As used herein, the term “avidity” refers to a total binding strength of a ligand with a receptor molecule, such that the strength of an interaction comprises multiple independent binding interactions between partners, which can be derived from multiple low affinity interactions or a small number of high affinity interactions.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

As used herein, the term “biopsy tissue” refers to a sample of tissue that is removed from a subject for the purpose of determining if the sample contains cancerous tissue. In some embodiment, biopsy tissue is obtained because a subject is suspected of having cancer. The biopsy tissue is then examined for the presence or absence of cancer.

As used herein, the term “carrier molecule” refers to any molecule that is chemically conjugated to a molecule of interest.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above. A “compound of the invention” refers to an miR or agonist of miR as described herein.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length. As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length. Of course, these fragments must be considered in the context of the type of nucleic acid being used or the size of the nucleic acid that is the starting nucleic acid.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit,” as used herein, refers to the ability of a compound, agent, or method to reduce or impede a described function, level, activity, rate, etc., based on the context in which the term “inhibit” is used. Preferably, inhibition is by at least 10%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor or target molecule.

A “receptor” or target molecule is a compound that specifically binds to a ligand.

A ligand or a receptor “specifically binds to” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “mass tag”, as used herein, means a chemical modification of a molecule, or more typically two such modifications of molecules such as peptides, that can be distinguished from another modification based on molecular mass, despite chemical identity.

The term “measuring the level of expression” or “determining the level of expression” as used herein refers to any measure or assay which can be used to correlate the results of the assay with the level of expression of a gene or protein of interest.

Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present.

Micro-RNAs are generally about 16-25 nucleotides in length. In one aspect, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

As used herein, the term “peptide ligand” (or the word “ligand” in reference to a peptide) refers to a peptide or fragment of a protein that specifically binds to a molecule, such as a protein, carbohydrate, and the like. A receptor or binding partner of the peptide ligand can be essentially any type of molecule such as polypeptide, nucleic acid, carbohydrate, lipid, or any organic derived compound. Specific examples of ligands are peptide ligands of the present inventions.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties. As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant adeno-associated viral (AAV) vector comprising a regulatory element active in muscle cells” refers to an AAV that has been constructed to comprise a new regulatory element to drive expression or tissue-specific expression in muscle of a gene of choice or interest. As described herein such a constructed vector may also contain at least one promoter and optionally at least one enhancer as part of the regulatory element, and the recombinant vector may further comprise additional nucleic acid sequences, including those for other genes, including therapeutic genes of interest.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, the term “subject at risk for PAD” refers to a subject with one or more risk factors for developing PAD. Risk factors include, but are not limited to, gender, age, genetic predisposition, environmental expose, and previous incidents of PAD, and lifestyle.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “transfection” is used interchangeably with the terms “gene transfer”, “transformation,” and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease, disorder, or condition for the purpose of decreasing the risk of developing pathology associated with the disease, disorder, or condition.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein a “nucleotide repeat disorder” (NRD) defines a set of genetic disorders associated with a 3 to 6 nucleotide repeat in a gene. For example a C9 expansion is repeat of (GGGGCC)n, wherein n is an integer greater than 2, typically ranging from 3-6.

As used herein the term “NRD repeat” is a nucleotide sequence that comprises a repeated 3 to 6 nucleotide sequence that has been associated with a nucleotide repeat disorders (NRDs).

### EMBODIMENTS

In one embodiment, the present invention is directed to composition and methods for diagnosing and treating nucleotide repeat disorders (NRDs) including for example, Huntington's disease (HD), myotonic dystrophy (DM1), and inherited forms of amyotrophic lateral sclerosis (ALS) and frontotemporal dementia (FTD). It has been reported that NRDs are associated with specific NRD repeat sequences. Applicants have discovered that while these diseases are associated with different NRD repeat sequences, each of these NDR repeats have high homology to known miRNAs. Accordingly, applicants believe each of the NRDs acts through a common mechanism wherein the NDR repeats acts as a sponge, binding up its complementary miRNA and preventing the miRNA from performing its normal function in the cell. In accordance with the present invention methods for detecting NRD patients are provided as well as methods for overcoming the effects of miRNA sponging.

In accordance with one embodiment a method of treating a nucleotide repeat disorder in a patient comprises detecting the presence of an NRD repeat in a biological sample recovered from the patient and treating those patients having an NRDR repeat by administering an effective amount of a therapeutic that enhances the activity of the miRNA that corresponds to the detected NRD repeat. More particularly, the “miRNA” that corresponds to the detected NRD repeat encompasses miRNA's that share at least 75% sequence identity over a 13 bp sequence of an NRD and in one embodiment over 85% sequence identity over a 14 bp sequence of an NRD.

In one embodiment the administered therapeutic comprises at least one miRNA, including pre-miRNA and mature miRNA, or a mimic thereof. “miRNA mimics” are chemically synthesized nucleic acid based molecules, preferably double-stranded RNAs which mimic mature endogenous miRNAs after transfection into cells.

miRNAs are transcribed by RNA polymerase II (pol II) or RNA polymerase III and arise from initial transcripts, termed primary miRNA transcripts (pri-miRNAs), that are generally several thousand bases long. Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

An administered miRNA may be the naturally occurring miRNA or it may be an analogue or homologue of the miRNA. In one aspect, the miRNA, or analogue or homologues, are modified to increase the stability thereof in the cellular milieu. In an alternative aspect the miRNA is encoded by an expression vector and may be delivered to the target cell in a liposome or microvesicle.

In one aspect, the administered therapeutic agent is an miRNA agonist. As used herein an miRNA agonist is a molecule or compound that enhances the expression, levels, or activity of a target miRNA. In one aspect, the agonist is a polynucleotide comprising a mature sequence of a miR or an active homolog or fragment thereof. In one aspect, the agonist is expressed from an expression construct.

In accordance with one embodiment a method of treating a nucleotide repeat disorder in a patient is provided. In one embodiment the method comprises a first step of screening a biological sample to identify the specific NRD repeat sequence. More particularly, a biological sample comprising cells is recovered and nucleic acid sequences are isolated from the cells of the sample and analyzed using standard techniques known to those skilled in the art to identify the presence of one or more specific NRDs. In one embodiment a patient diagnosed with or suspected of having ALS will be screened for the presence of a C9 expansion repeat (SEQ ID NO: 6). The biological sample in one embodiment is a blood sample or derivative thereof, or a tissue or urine sample.

In one embodiment the step of detecting the NRD repeat comprises contacting nucleic acids recovered from a biological sample with a reagent that specifically binds to an NRD repeat. In one embodiment the nucleic acids recovered from the patient's biological sample is amplified using PCR. In one embodiment the NRD repeat is detected using a labeled oligonucleotide that is complementary to the NRD repeat. In one embodiment the patient's nucleic acid sequences are screened for the presence of an NDR repeat comprising the sequence of SEQ ID NO: 2, SEQ ID NO: 4, SEQ ID NO: 6, SEQ ID NO: 8 or SEQ ID NO: 10. In one embodiment the oligonucleotide used to detect the NDR repeat is a sequence selected from SEQ ID NO: 1, SEQ ID NO: 3, SEQ ID NO: 5, SEQ ID NO: 7, or SEQ ID NO: 9 or complement thereof.

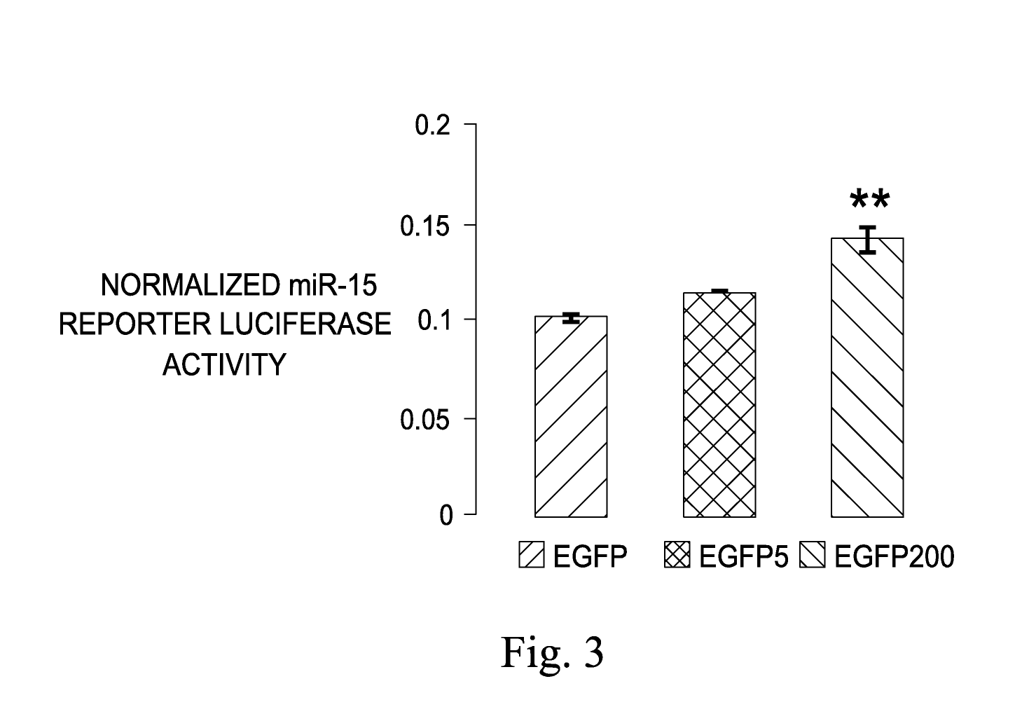

Those patients that are identified as containing an NRD repeat will then be treated with a therapeutic regiment that enhances the activity of the miRNA that corresponds to the detected NRD repeat. In one embodiment the therapeutic regiment enhances the activity of an miRNA selected from the group consisting of SEQ ID NO: 1, SEQ ID NO: 3, SEQ ID NO: 5, SEQ ID NO: 7, and SEQ ID NO: 9. As used herein the “activity” of an miRNA is determined by its ability to inhibit the expression of a marker gene in an in vitro assay as disclosed herein (see Example 2; FIG. 3)

In one embodiment the therapeutic regiment comprises introducing nucleic acid sequences that increase the cellular concentration of the miRNA that corresponds to the detected NRD repeat. In an alternative embodiment the therapeutic regiment comprises administering a pharmaceutical agent that enhances the cellular concentration of the miRNA that corresponds to the detected NRD repeat. In one embodiment the pharmaceutical agent is an HDAC inhibitor, including for example an HDAC inhibitor selected from the group consisting of trichostatin A, valproate and vorinostat. In one embodiment the HDAC inhibitor is valproate or trichostatin A. In one embodiment the HDAC inhibitor is valproate. In one embodiment the HDAC inhibitor is trichostatin A. In one embodiment the patient is administered a pharmaceutical composition comprising 2 or more HDAC inhibitors selected from the group consisting of trichostatin A, valproate and vorinostat. In one embodiment the patient is administered a pharmaceutical composition comprising trichostatin A and valproate.

In accordance with one embodiment a method of treating inherited forms of amyotrophic lateral sclerosis (ALS) and frontotemporal dementia (FTD) in a patient is provided. The method comprises screening patients for the presence of a C9 expansion sequence comprising SEQ ID NO: 6 in a biological sample recovered from said patient; and treating patients having said C9 expansion with a therapeutic agent that
a) enhances activity of miR-762, and/or
b) decreases activity of NETO1.

In one embodiment the therapeutic agent enhances the activity of miR-762, as measured by the assay of Example 2, optionally by increasing the effective concentration of miR-762. In one embodiment the effective concentration of miR-762 is increased by administering a viral vector (e.g. a lentivirus) comprising and miR-762 encoding sequence.

In one embodiment the activity of miR-762 is enhanced in the cells of the patient by administering an HDAC inhibitor, including for example an HDAC inhibitor selected from the group consisting of trichostatin A, valproate and vorinostat. Administration can be by any standard route including oral administration. In one embodiment the HDAC inhibitor is valproate or trichostatin A. In one embodiment the HDAC inhibitor is valproate. In one embodiment the HDAC inhibitor is trichostatin A. In one embodiment the patient is administered a pharmaceutical composition comprising 2 or more HDAC inhibitors selected from the group consisting of trichostatin A, valproate and vorinostat. In one embodiment the patient is administered a pharmaceutical composition comprising trichostatin A and valproate and a pharmaceutically acceptable carrier.

In accordance with one embodiment the activity of NETO1 is decreased in the cells of the patient by introducing an interfering RNA or an antisense nucleic acid sequence. In one embodiment the activity of NETO1 is decreased by enhancing the activity of miR-762.

In one embodiment a kit is provided for detecting patients having an NRD and comprising and identifying the NRD repeat associated with the NRD. In accordance with one embodiment the kit comprises an oligonucleotide that specifically binds to a nucleic acid, or a corresponding compliment thereof, selected from the group consisting of SEQ ID NO: 2, SEQ ID NO: 4, SEQ ID NO: 6, SEQ ID NO: 8 and SEQ ID NO: 10. The kit may contain additional instructions materials and vials and vessels for conducting reactions. In one embodiment the kit comprises reagents for detecting the binding of the oligonucleotide to its target sequence. In accordance with one embodiment the kit comprises a nucleic acid selected from the group consisting of SEQ ID NO: 1, SEQ ID NO: 3, SEQ ID NO: 5, SEQ ID NO: 7, and SEQ ID NO: 9. In one embodiment the kit further comprises an HDAC inhibitor selected from the group consisting of trichostatin A, valproate and vorinostat.

In one embodiment, the agonist is administered to a subject by intravenous injection. In one aspect, the agonist is administered directly to the site of the disease, disorder or condition and the associated ischemia. In one embodiment, an miR-specific inhibitor/antagonist is an anti-miRNA (anti-miR) oligonucleotide (for example, see WO2005054494).

In one embodiment, the agonist is administered to a subject by oral, intravenous, intramuscular, transdermal, sustained release, controlled release, delayed release, suppository, subcutaneous, catheter, topical, or sublingual administration.

The term “expression construct” is meant to include any type of genetic construct containing a nucleic acid coding for a gene product in which part or all of the nucleic acid encoding sequence is capable of being transcribed. Generally, the nucleic acid encoding a gene product is under transcriptional control of a promoter. A “promoter” refers to a DNA sequence recognized by the synthetic machinery of the cell, or introduced synthetic machinery, required to initiate the specific transcription of a gene.

In one embodiment, a vector of the invention is a viral vector. In one aspect, the vector is an AAV (adeno-associated virus) vector. In one embodiment, a recombinant AAV vector of the invention is useful for targeting muscle preferentially over other tissues. In one embodiment, a recombinant AAV vector of the invention is useful for increasing expression of a gene of interest preferentially in muscle. The compositions and methods disclosed herein encompass targeting and transducing muscle with an AAV vector. The method comprises administering to a subject a pharmaceutical composition comprising an effective amount of a recombinant adeno-associated viral (AAV) vector comprising a regulatory element. The regulatory element comprises at least one promoter element and optionally at least one enhancer element, wherein the enhancer and promoter are operably linked. The recombinant AAV vector also may optionally comprise at least one gene operably linked to a promoter element. The AAV may comprise the entire AAV genome, or a homolog or fragment thereof, such as the capsid of the particular AAV. However, it should be noted that the entire AAV genome may not be useful in some situations because of a need to make the vector replication-deficient and/or to insert genes of interest such as therapeutic genes.

The regulatory elements and the gene of interest may also be substituted with active fragments, modifications, or homologs thereof. In one aspect, the recombinant AAV vector preferentially targets skeletal muscle.

A recombinant AAV vector can be prepared for use in knocking down specific genes in muscle with siRNA or miRNA expressed from an AAV vector of the invention.

Other useful vectors, nucleic acids, and proteins or homologs and fragments thereof are useful with the practice of the invention, including but not limited to AAV-9-NCBI Accession number AX753250 and AAV-8-NCBI Accession number NC 006261.

Due to the payload constraints of AAV, in one embodiment a cDNA may be preferred. In one aspect, additional introns and sequences can be introduced. In one aspect, the cap gene of the AAV is used and not the entire AAV genomic DNA.

Other methods and vectors are known in the art which could also be used to practice the methods of the present invention, including those in Souza et al. (U.S. Pat. Pub. No. 2011/0212529, published Sep. 1, 2011).

Although AAVs such as AAV9 and AAV8 may target some tissues with higher specificity than other tissues, the use of tissue or cell specific enhancers and promoters as part of the vector can help to ensure that the genes of interest are expressed in the desired cell or tissue. A more detailed description and use of AAVs can be found in U.S. Pat. Pub. No. US 2013/0136729 (French and Annex, U.S. patent application Ser. No. 13/673,351), the entirety of which is incorporated by reference herein.

Ordahl et al. (U.S. Pat. No. 5,266,488) characterized the chicken troponin-T gene promoter and found the essential proximal promoter element contains nonspecific sequences necessary for the initiation of transcription of a structural gene to be operatively associated with the promoter. When +1 designates the first nucleotide of the transcription initiation site, this element is located between nucleotide −49 and nucleotide +1. Further, Ordahl demonstrated that the skeletal muscle-specific regulatory element is positioned upstream of the essential proximal promoter element and is operationally associated therewith. This element is necessary for the expression of a structural gene to be operatively associated with the promoter in skeletal muscle cells. The skeletal muscle-specific regulatory element is located between nucleotide −129 and −49. Ordahl also stated that the cardiac muscle-specific regulatory element is positioned upstream of both the skeletal muscle specific regulatory element and the essential proximal promoter element and is operatively associated with the essential proximal promoter element and suggested that this element is necessary for the expression of a structural gene to be operatively associated with the promoter in cardiac muscle cells. Ordahl also asserted that the cardiac muscle-specific regulatory element is located between nucleotide −268 and nucleotide −201.

Ordahl also demonstrated that the nonessential positive striated muscle regulatory element is positioned upstream of, and operationally associated with, both the skeletal muscle specific regulatory element and the cardiac muscle-specific regulatory element. This element facilitates the expression of a structural gene to be operatively associated with the promoter in striated muscle cells, both cardiac and skeletal. This element is located between nucleotide −550 and −269.

According to Ordahl, the nonessential negative regulatory element is positioned upstream of the positive striated muscle regulatory element and is operatively associated therewith. This element inhibits the positive striated muscle regulatory element from facilitating the expression of a structural gene to be operatively associated with the promoter. This element is located between nucleotide −3000 and nucleotide −1100. More broadly defined, this element is located between nucleotide −3000 and nucleotide −550.

In one embodiment, the present invention encompasses the use of the promoter regions described by Ordahl for targeting muscle in general or for more specifically targeting cardiac muscle over skeletal muscle or vice-versa.

A complete promoter (one containing all the elements described above) expresses a structural gene operatively associated therewith in both skeletal and striated muscle cells. The individual elements which comprise a complete promoter can be used in any desired operable combination to produce new promoters having different properties. For example, the negative nonspecific regulatory element can be deleted from a complete promoter so that the expression of a gene associated with the promoter is facilitated. The cardiac muscle-specific regulatory element can be deleted from a complete promoter so that a structural gene operatively associated with the promoter is preferentially expressed in skeletal cells, or the skeletal muscle-specific regulatory element can be deleted from a complete promoter so that a structural gene operatively associated with the promoter is preferentially expressed in cardiac cells. The term “deleted,” as used herein, means any modification to a promoter element which renders that element inoperable.

Operable promoters can be constructed from the minimum necessary regulatory elements. One such promoter comprises an essential proximal promoter element and a cardiac muscle-specific regulatory element positioned upstream of the essential proximal promoter element and operatively associated therewith. Another such promoter comprises an essential proximal promoter element and a skeletal muscle-specific regulatory element positioned upstream of said essential proximal promoter element and operatively associated therewith. To these promoters, a positive striated muscle regulatory element may optionally be positioned upstream oft and operatively associated with, the specific regulatory element (skeletal or cardiac).

Therefore, the present invention encompasses the use of a cardiac troponin-T promoter, for example, where the sequence comprises a promoter and is the 5′ region of about nucleotide position −3000 to about the transcription start site of cardiac troponin-T or about nucleotide +25 to about +50, or where the sequence comprises the 5′ region of about nucleotide −1000 to about the transcription start site or about nucleotide +25 to about +50, or where the sequence comprises the 5′ region of about nucleotide −550 to about the transcription start site or about nucleotide +25 to about +50, or where the sequence comprises the 5′ region of about nucleotide −400 to about the transcription start site or about nucleotide +25 to about +50, or where the sequence comprises the 5′ region of about nucleotide −300 to about the transcription start site or about nucleotide +25 to about +50. In one aspect, the sequence is about 375 nucleotides upstream (−) to 43 nucleotides downstream (+) (see Example 1). In another aspect, the sequence is 5′ region from about nucleotide −268 to about nucleotide +38 relative to the transcription start site.

It will be understood by one of ordinary skill in the art that when a different promoter is being used, such as a muscle creatine kinase promoter, similar to the cardiac troponin-T promoter various lengths of the sequence can also be used.

In one embodiment, the present invention encompasses compositions and methods for transducing skeletal muscle and enhancing gene expression using an AAV vector engineered to comprise a skeletal muscle gene promoter. In one aspect, the AAV is AAV9 or AAV8.

In one embodiment, the present invention relates to gene therapy methods utilizing tissue-specific expression vectors. The invention further relates to expression vectors used for delivery of a transgene into muscle. In one aspect, the muscle is cardiac muscle. In another aspect, the muscle is skeletal muscle. More specifically, the invention relates to transcriptional regulatory elements that provide for enhanced and sustained expression of a transgene in the muscle.

Skeletal muscle promoters and enhancers are available for the muscle creatine kinase (MCK) gene and are encompassed by the presented invention for regulating expression of a therapeutic gene in an AAV vector of the invention.

Accordingly, one embodiment of the invention provides expression vectors optimized for sustained expression of a transgene in muscle tissue. Another object of this invention is to provide enhancer/promoter combinations that can direct sustained and appropriate expression levels in various expression systems.

In one embodiment, the invention encompasses combining minimal sequences from muscle-specific promoters and muscle-specific enhancers to create chimeric regulatory elements that drive transcription of a transgene in a sustained fashion. A minimal sequence is one which maintains the function of interest, although possibly somewhat less than the full sequence of interest. The resulting chimeric regulatory elements are useful for gene therapy directed at transgene expression in the muscle as well as other applications requiring long-term expression of exogenous proteins in transfected muscle cells such as myocytes. In one aspect, the myocytes are cardiac myocytes. In another aspect, the myocytes are skeletal muscle myocytes.

Chimeric regulatory elements useful for targeting transgene expression to the muscle are provided by the invention. The chimeric regulatory elements of the invention comprise combinations of muscle-specific promoters and muscle-specific enhancers that are able to direct sustained transgene expression preferentially in the muscle. In one aspect, the enhancers and promoters are cardiac specific and in another aspect, the enhancers and promoters are skeletal muscle specific.

The present invention is also directed to recombinant transgenes which comprise one or more operably linked tissue-specific regulatory elements of the invention. The tissue-specific regulatory elements, including muscle-specific promoter and enhancers operably linked to a transgene, drive its expression in myocytes and, in particular, in cardiomyocytes and/or skeletal myocytes. The transgenes may be inserted in recombinant viral vectors for targeting expression of the associated coding DNA sequences in muscle. Muscle-specific promoters useful in the invention include, for example, muscle creatine kinase (MCK) promoter, cardiac troponin-T promoter, or desmin (DES) promoter. In one particular embodiment, the promoter is a human promoter. In another embodiment, the promoter is a murine promoter. In yet another embodiment, the promoter is a chicken promoter. In certain embodiments, the promoter is truncated.

In one embodiment, tissue-specific enhancers are used. Tissue-specific enhancers include muscle specific enhancers. One or more of these muscle-specific enhancer elements may be used in combination with a muscle-specific promoter of the invention to provide a tissue-specific regulatory element. In one embodiment, the enhancers are derived from human, chicken, or mouse. In certain embodiments, the enhancer/enhancer or enhancer/promoter combinations are heterologous, i.e., derived from more than one species. In other embodiments, the enhancers and promoters are derived from the same species. In certain embodiments, enhancer elements are truncated.

In one embodiment, a regulatory element of the invention comprises at least one MCK or cardiac troponin-T enhancer operably linked to a promoter. In another embodiment, a regulatory element of the invention comprises at least two MCK enhancers linked to a MCK promoter or a DES promoter or a cardiac troponin-T promoter. In yet another embodiment, a regulatory element comprises at least two DES enhancers linked to a promoter. In a further embodiment, a regulatory element comprises at least two cardiac troponin-T enhancers linked to a promoter.

The invention provides vectors comprising a regulatory element of the invention. In some embodiments, a regulatory element of the invention is incorporated into a viral vector such as one derived from adenoviruses, adeno-associated viruses (AAV), or retroviruses, including lentiviruses such as the human immunodeficiency (HIV) virus. In one embodiment, the AAV is AAV8 or AAV9. The invention also encompasses methods of transfecting muscle tissue where such methods utilize the vectors of the invention.

The invention further provides cells transfected with the nucleic acid containing an enhancer/promoter combination of the invention.

Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis as the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., J. Bio. Chem. 1990, 266: 6562-6570). Furthermore, unlike promoter elements, enhancers can be active when placed downstream from the transcription initiation site, e.g., within an intron, or even at a considerable distance from the promoter (Yutzey et al., Mol. and Cell. Bio. 1989, 9:1397-1405).

It is known in the art that some variation in this distance can be accommodated without loss of promoter function. Similarly, the positioning of regulatory elements with respect to the transgene may vary significantly without loss of function. Multiple copies of regulatory elements can act in concert. Typically, an expression vector comprises one or more enhancer sequences followed by, in the 5′ to 3′ direction, a promoter sequence, all operably linked to a transgene followed by a polyadenylation sequence.

The present invention further relies on the fact that many enhancers of cellular genes work exclusively in a particular tissue or cell type. In addition, some enhancers become active only under specific conditions that are generated by the presence of an inducer such as a hormone or metal ion. Because of these differences in the specificities of cellular enhancers, the choice of promoter and enhancer elements to be incorporated into a eukaryotic expression vector is determined by the cell type(s) in which the recombinant gene is to be expressed.

In one aspect, the regulatory elements of the invention may be heterologous with regard to each other or to a transgene, that is, they may be from different species. Furthermore, they may be from species other than the host, or they also may be derived from the same species but from different genes, or they may be derived from a single gene.

The present invention further includes the use of desmin regulatory elements. Desmin is a muscle-specific cytoskeletal protein that belongs to the family of intermediate filaments that occur at the periphery of the Z disk and may act to keep adjacent myofibrils in lateral alignment. The expression of various intermediate filaments is regulated developmentally and shows tissue specificity.

The muscle creatine kinase (MCK) gene is highly active in all striated muscles. Creatine kinase plays an important role in the regeneration of ATP within contractile and ion transport systems. It allows for muscle contraction when neither glycolysis nor respiration is present by transferring a phosphate group from phosphocreatine to ADP to form ATP. There are four known isoforms of creatine kinase: brain creatine kinase (CKB), muscle creatine kinase (MCK), and two mitochondrial forms (CKMi). MCK is the most abundant non-mitochondrial mRNA that is expressed in all skeletal muscle fiber types and is also highly active in cardiac muscle. The MCK gene is not expressed in myoblasts, but becomes transcriptionally activate when myoblasts commit to terminal differentiation into myocytes. MCK gene regulatory regions display striated muscle-specific activity and have been extensively characterized in vivo and in vitro. Mammalian MCK regulatory elements are described, for example, in Hauser et al., Mol. Therapy 2000, 2:16-25 and in Souza et al., 2011. MCK enhancer and promoter sequences are provided herein.

The present invention further includes the use of troponin regulatory elements, particularly cardiac troponin.

The present invention further includes the use of combinations of elements to form, for example, chimeric regulatory elements. The present invention is directed to recombinant transgenes which comprise one or more of the tissue-specific regulatory elements described herein. The chimeric tissue-specific regulatory elements of the invention drive transgene expression in muscle cells. In one aspect the muscle cell is a skeletal muscle cell. In one aspect, the muscle cell is a cardiomyocyte. The transgenes may be inserted in recombinant viral or non-viral vectors for targeting expression of the associated coding DNA sequences in muscle. In one aspect, the viral vector is an AAV. In one embodiment, the promoter element is selected from the group consisting of muscle creatine kinase (MCK) promoter, desmin promoter, and cardiac troponin T promoter. In one particular embodiment, the promoter is a human promoter. In another embodiment, the promoter is a murine promoter. In another embodiment, the promoter is a chicken promoter. In certain embodiments, the promoter is truncated. One of ordinary skill in the art will appreciate that the entire promoter need not necessarily be used in all cases and that activity can be maintained when some nucleotides are deleted or added.

It will be understood that the regulatory elements of the invention are not limited to specific sequences referred to in the specification but also encompass their structural and functional analogs/homologues and functional fragments thereof. Such analogs may contain truncations, deletions, insertions, as well as substitutions of one or more nucleotides introduced either by directed or by random mutagenesis. Truncations may be introduced to delete one or more binding sites for known transcriptional repressors. Additionally, such sequences may be derived from sequences naturally found in nature that exhibit a high degree of identity to the sequences in the invention. In one aspect, a nucleic acid of 20 nt or more will be considered to have high degree of identity to a promoter/enhancer sequence of the invention if it hybridizes to such promoter/enhancer sequence under stringent conditions. Alternatively, a nucleic acid will be considered to have a high degree of identity to a promoter/enhancer sequence of the invention if it comprises a contiguous sequence of at least 20 nt, which has percent identity of at least 60%, 65%, 70%, 75%, 80%, 85%, 90%, 95%, or more as determined by standard alignment algorithms such as, for example, Basic Local Alignment Tool (BLAST) described in Altschul et al., J. Mol. Biol. 1990, 215: 403-410, the algorithm of Needleman et al., J. Mol. Biol. 1970, 48: 444-453, or the algorithm of Meyers et al., Comput. Appl. Biosci. 1988, 4: 11-17. Non-limiting examples of analogs, e.g., homologous promoter sequences and homologous enhancer sequences derived from various species, are described in the present application.

In one embodiment, the invention further includes vectors comprising a regulatory element of the invention. In general, there are no known limitations on the use of the regulatory elements of the invention in any vector. A regulatory element comprises a promoter element and optionally an enhancer element.

In one embodiment an antagonist of miR may be used. In one aspect, the antagonist is an antisense oligonucleotide or an antagomir. In one aspect, the antisense oligonucleotide comprises a sequence that is at least partially complementary to a mature sequence of an miR.

miRNA expression vectors are known in the art, for example, from: Cell Biolabs (RAPAd® miRNA Adenoviral Expression System, Cat. # VPK-253; pMXs-miR-GFP/Puro Retroviral Expression Vector Cat. # RTV-017; miRNASelect™ pEGP-miR Cloning & Expression Vector, Cat. # MIR-EXP-GP-C; miRNASelect™ pEP-miR Cloning & Expression Vector, Cat. # MIR-EXP-C); SBI's (System Biosciences) lentivector systems; Clontech; Origene's MicroRNA eXpression plasmid for over-expression of miRNAs of choice (##'s SC410001 and SC410002); Life Technologies/Ambion (multiple vectors, including for control miRNAs).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-1-stranded DNA, supercoiled DNA and/or triple-helical DNA; Z-DNA; miRNA, siRNA, and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

miRNAs are RNA molecules of about 22 nucleotides or less in length, but are variable in length. These molecules are post-transcriptional regulators that bind to complementary sequences on target mRNAs. Although miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. In one embodiment, an anti-miRNA oligomer directed against an miR can be used. See U.S. patent application Ser. No. 13/503,189, WO2007/112754, and WO2007/112653 for additional descriptions of oligomers, locked nucleic acid oligomers, gapmers, mixmers, totalmers, etc. In one aspect, an anti-miR-93 can be purchased.

The invention is also directed to methods of administering the compounds, cells, proteins and peptides (collectively referred to as compounds) of the invention to a subject.

In one aspect the nucleic acid is an antisense molecule, an oligonucleotide, an RNA, an siRNA, and an miRNA.

Although miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules.

Pharmaceutical compositions comprising the present compounds are administered to an individual in need thereof by any number of routes which effectively transport the active compound to the appropriate or desired site of action including, but not limited to, topical, oral, intravenous, intramuscular, intra-arterial, intramedullary, intrathecal, intraventricular, parenteral, transdermal, subcutaneous, intraperitoneal, intranasal, enteral, topical, sublingual, pulmonary, buccal, vaginal, or rectal means.

The present invention is also directed to pharmaceutical compositions comprising the polynucleotides/nucleic acids of the present invention. More particularly, such compounds can be formulated as pharmaceutical compositions using standard pharmaceutically acceptable carriers, fillers, solublizing agents and stabilizers known to those skilled in the art.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

In one embodiment, the pharmaceutical compositions useful for practicing the invention may be administered to deliver a dose of nucleic acid or additional therapeutic agent of between 1 ng/kg/day and 100 mg/kg/day. Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a subject for treatment of the diseases disclosed herein.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A composition of the invention may comprise additional ingredients. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical composition may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the condition or disease being treated, the type and age of the animal, etc.

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies.

Additional Therapeutic Agents and Ingredients

The composition of the invention can further comprise additional therapeutic additives, alone or in combination (e.g., 2, 3, or 4 additional additives). Examples of additional additives include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; (e) growth factors; (f) cytokines; (g) hormones; and (h) combinations thereof.

In one embodiment, a formulation of the invention contains an antimicrobial agent. The antimicrobial agent may be provided at, for example, a standard therapeutically effective amount. A standard therapeutically effective amount is an amount that is typically used by one of ordinary skill in the art or an amount approved by a regulatory agency (e.g., the FDA or its European counterpart). Antimicrobial agents useful for the invention include those directed against the spectrums of gram positive organisms, gram negative organisms, fungi, and viruses.

According to the topical anesthetic embodiment of the present invention, in one aspect, suitable local anesthetic agents having a melting point of 30° to 70° C. are prilocaine, tetracaine, butanilcaine, trimecaine, benzocaine, lidocaine, bupivocaine, dibucaine, mepivocaine, and etidocaine.

The present invention further encompasses the use of at least two anesthetics.

The local anesthetic composition of the present invention may further comprise suitable additives, such a pigment, a dye, an anti-oxidant, a stabilizer or a fragrance provided that addition of such an additive does not destroy the single phase of the anesthetic composition.

In one aspect, the hydrated local anesthetic mixture is prepared by melting the local anesthetic with the higher melting point of the two, followed by addition of the other local anesthetic, under vigorous mechanical mixing, such as trituration or grinding. A milky viscous liquid is formed, at which point, the surfactant is added with more mechanical mixing. Mixing of the surfactant produces a milky liquid of somewhat lower viscosity. Finally, the balance of water is added under vigorous mechanical mixing. The material can then be transferred to an air tight container, after which a clear composition is obtained after about 60 minutes at room temperature.

Alternatively, the hydrated local anesthetic mixture can be prepared by first melting the lower melting local anesthetic, followed by addition of the other local anesthetic along with vigorous mechanical mixing, then addition of the surfactant and water as above. However, when the lower melting local anesthetic is melted first, the storage time needed to obtain the single phase composition, increases from about 1 hour to about 72 hours. Accordingly, the former method is preferred.

One of ordinary skill in the art will appreciate that there are multiple suitable surfactants useful for preparing the hydrated topical anesthetic of the present invention. For example, single-phase hydrated topical anesthetics can be prepared from anionic, cationic or non-ionic surfactants.

Several preferred embodiments include use of any therapeutic molecule including, without limitation, any pharmaceutical or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfiram and disulfiram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present invention.

A list of the types of drugs, and specific drugs within categories which are encompassed within the invention is provided below and are intended be non-limiting examples.

Antimicrobial Agents Include:

silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), Neosporin (i.e., Bacitracin, Polymyxin B, and Neomycin), Polysporin (i.e., Bacitracin and Polymyxin B). Additional antimicrobials include topical antimicrobials (i.e., antiseptics), examples of which include silver salts, iodine, benzalkonium chloride, alcohol, hydrogen peroxide, and chlorhexidine.

Analgesic:

Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

Antihypertensive:

Aflyzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Mono sulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride; Pazoxide; Pelanserin Hydrochloride; Perindopril Erbumine; Phenoxybenzamine Hydrochloride; Pinacidil; Pivopril; Polythiazide; Prazo sin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazo sin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine.

Anti-inflammatory:

Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Growth Factors

In one embodiment, an effective amount of at least one growth factor, cytokine, hormone, or extracellular matrix compound or protein useful for enhancing wound healing is administered. In one aspect, a combination of these agents is used. In one aspect, growth factors useful in the practice of the invention include, but are not limited to, EGF, PDGF, GCSF, IL6, IL8, IL10, MCP1, MCP2, Tissue Factor, FGFb, KGF, VEGF, PLGF, MMP1, MMP9, TIMP1, TIMP2, TGFβ, and HGF. One of ordinary skill in the art will appreciate that the choice of growth factor, cytokine, hormone, or extracellular matrix protein used will vary depending on criteria such as the type of injury, disease, or disorder being treated, the age, health, sex, and weight of the subject, etc. In one aspect, the growth factors, cytokines, hormones, and extracellular matrix compounds and proteins are human.

Proteins and other biologically active compounds that can be incorporated into, or included as an additive within, a composition comprising compounds of the present invention include, but are not limited to, collagen (including cross-linked collagen), fibronectin, laminin, elastin (including cross-linked elastin), osteopontin, osteonectin, bone sialoproteins (Bsp), alpha-2HS-glycoproteins, bone Gla-protein (Bgp), matrix Gla-protein, bone phosphoglycoprotein, bone phosphoprotein, bone proteoglycan, protolipids, bone morphogenetic protein, cartilage induction factor, skeletal growth factor, enzymes, or combinations and biologically active fragments thereof. Adjuvants that diminish an immune response can also be used in conjunction with the composite of the subject invention.

Other molecules useful as compounds or substances in the present invention include, but are not limited to, growth hormones, leptin, leukemia inhibitory factor (LIF), tumor necrosis factor alpha and beta, endostatin, angiostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12,13, 15, 16, 17 and 18. Embodiments involving amino acids, peptides, polypeptides, and proteins may include any type of such molecules of any size and complexity as well as combinations of such molecules.

Other embodiments of the invention will be apparent to those skilled in the art based on the disclosure and embodiments of the invention described herein. It is intended that the specification and examples be considered as exemplary only, with a true scope and spirit of the invention being indicated by the following claims. While some representative experiments have been performed in test animals, similar results are expected in humans. The exact parameters to be used for injections in humans can be easily determined by a person skilled in the art.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### Example 1

### Assessing NRD Nucleotide Repeat Expansion for Potential miR Homology

Validated miR target prediction software will be used to assess each NRD nucleotide repeat expansion for potential miR homology. Both sense and antisense repeat transcripts will be used as inputs, based on reports suggesting that antisense transcripts are produced in these disorders. Some results of this analysis are shown in Table 1. Every single NRD expansion provides a predicted seed match for at least one miR—and in most cases only one—from the forward (sense) or reverse (antisense) transcript. The striking degree of seed match homology and the one-to-one expansion-to-miR ratio strongly support our hypothesis and argue for their relevance.

Several of the miR matches are present in cell types affected by the expanded repeats and/or have potentially relevant roles. For C9ORF72, a very striking match between the antisense repeat and miR-762 was identified. Importantly, the C9 reverse transcript is as prevalent as the forward transcript in affected brain regions. While little is known of miR-762, it has been linked to neural stem cell (NSC) differentiation. DM1 and HD share a sense/antisense repeat, and applicants have identified a seed match to the miR-15 family. This miR family has also been linked to NSC differentiation, and cell differentiation defects have been identified as a feature of DM1 pathology. Though

TABLE 1
Sample of NRD-miR pair alignments

| | Expanded | Sense/anti- | miR | | |
|---|---|---|---|---|---|
| NRD | repeat | sense | homology | Sequence alignment | |
| DM1 | (CTG)n | Sense | miR-15 | miRNA: | 3′ guguuUGGUAAU-AC-ACGACGAu 5′ |
| HD | | Antisense | family | | :\|:: \|: \|\| \|\|\|\|\|\|\|: |
| | | | | Target: | 5′ ---cuGCUGCUGCUGCUGCUGCUg 3′ |
| FXS | (CGG)n | Sense | miR-1181 | miRNA: | 3′ gcCGAGCCCACCGCCGCUGCc 5′ |
| FXTAS | | | | | \|\|\|\| \|\| \|\|\|\|\|\|\|:\|\|\| |
| | | | | Target: | 5′ cgGCGGCGGCGGCGGCGGCGg 3′ |
| FTD- | (GGGGCC)n | Antisense | miR-762 | miRNA: | 3′ cgaGCCGGGGCCGGGGUCGGGg 5′ |
| MND | | | | | \| \|\|\|\|\|\|\|\|\|\|\|\|\|:\|\|\|\|\| |
| | | | | Target: | 5′ cccCGGCCCCGGCCCCGGCCCc 3′ |
| SCA10 | (ATTGT)n | Sense | miR-374B | miRNA: | 3′ gugaAUCGUCCAACAUAAUAUa 5′ |
| | | | | | \| \|:\| \|\|\|\|\|\|\|:\|\|\| |
| | | | | Target: | 5′ ---aUUGUA--UUGUAUUGUAu 3′ |
| EPM1* | (CCCCGCCC | Sense | miR-663 | miRNA: | 3′ cgcCAGGGC--GCCGCGGGGCGGa 5′ |
| | CGCG)n | | | | \| \|\|\|\| \| \|\|\|\|\|\|\|\|\|\| |
| | | | | Target: | 5′ cgcGCCCCGCCCCGCGCCCCGCCc 3′ |

*myoclonic epilepsy of Unverricht-Lundborg type
Table 1: Sample of NRD-miR pair alignments DM1 HD (miRNA: SEQ ID NO: 1; Target: SEQ ID NO: 2); FXS FXTAS (miRNA: SEQ ID NO: 3; Target: SEQ ID NO: 4); FTD-MND (miRNA: SEQ ID NO: 5; Target: SEQ ID NO: 6); SCA10 (miRNA: SEQ ID NO: 7; Target: SEQ ID NO: 8); EPM1 (miRNA: SEQ ID NO: 9; Target: SEQ ID NO: 10)

the sponging hypothesis might be interpreted to suggest identical manifestations in diseases sharing a repeat motif, the diseases present very differently. This could be explained by differences in timing, pattern, and location of repeat region transcript expression in different cells. Disrupted miR expression also fits well with the slowly progressive nature of most of these diseases.

### Example 2

### In Vitro Sponging Capabilities of Two Nucleotide Repeat Expansions (NREs)

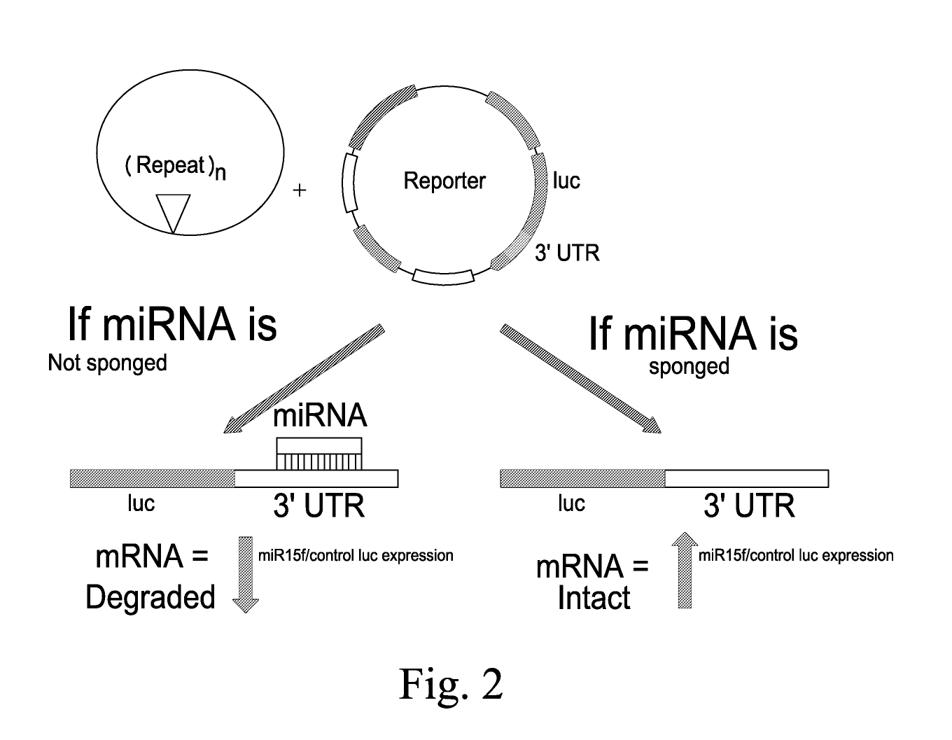

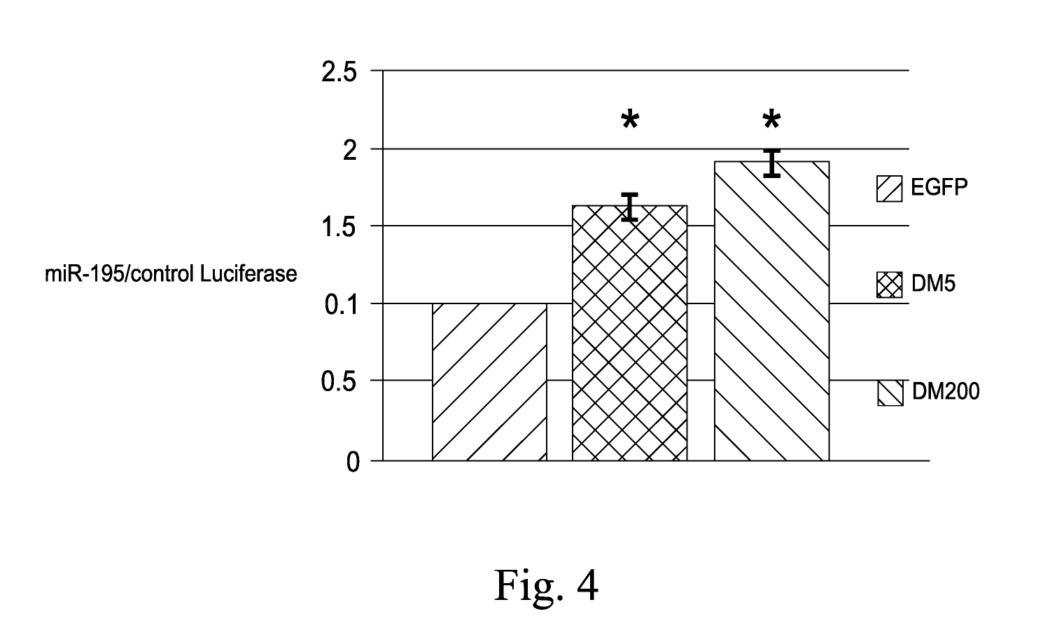

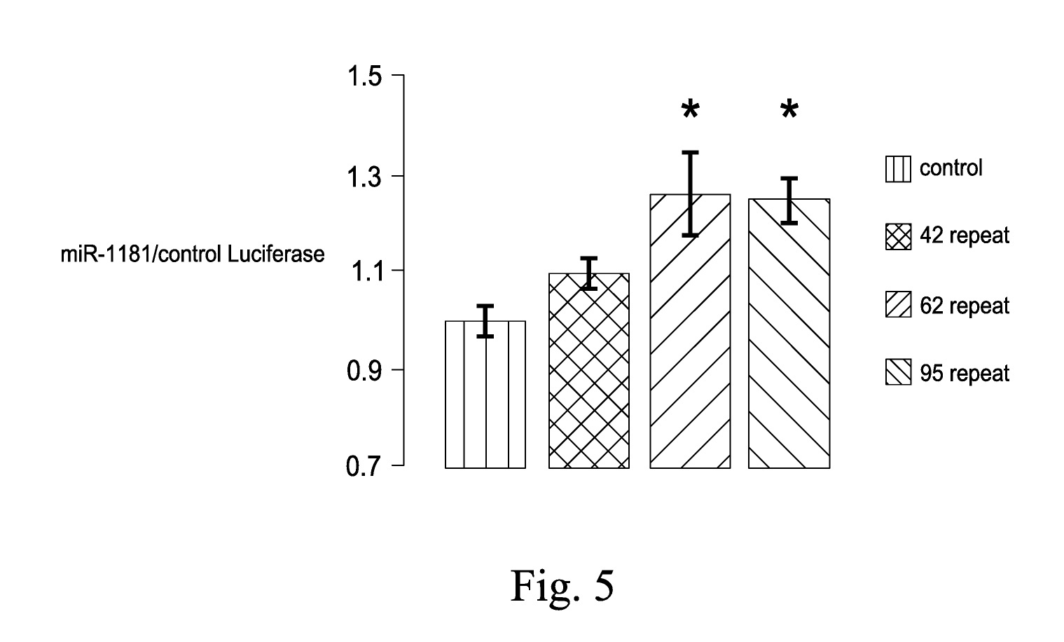

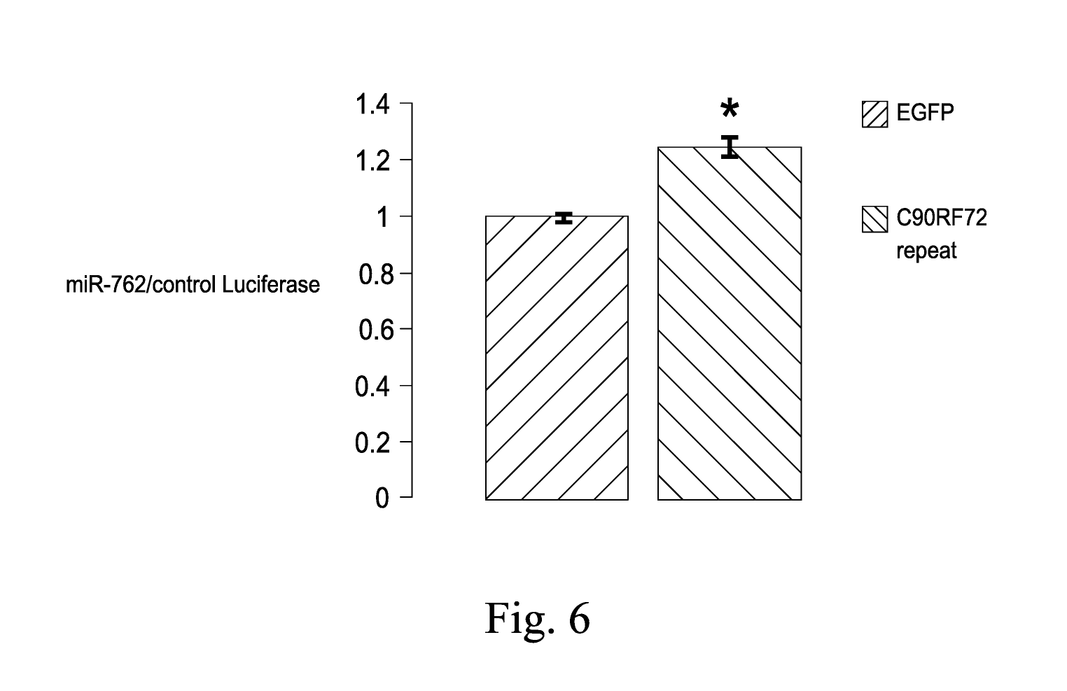

To determine whether miR sponging by nucleotide repeat expansions can occur in vitro, miR-specific luciferase-based reporter assays were used (FIG. 2). Cells were co-transfected with either an miR-15 reporter+(CTG)n (FIG. 3), a miR-195 reporter+(CUG)n (representing the DM1/HD disease state) (FIG. 4), a miR-1181 reporter+(CGG)n (FIG. 5), or a miR-762 reporter+(CCCCGG)12 (for FTD-MND) (FIG. 6). Controls include both nonspecific luciferase reporter and an empty vector for each experiment to control for non-specific reporter activity. As shown in FIGS. 3-6, increased expression of luciferase in the presence of the expanded repeats was demonstrated, indicating reduced cellular miR activity—consistent with sponging. Overall miR levels were not altered, arguing against a degradation effect. These data suggest that miR sponging may occur across multiple NRD's and also that the sponged miRs predicted via the in silico analysis are valid.

### Example 3

### Mir-762 Levels can be Altered by HDAC Inhibitors

Some miR-15 family members are up-regulated by demethylating drugs and histone deacetylase (HDAC) inhibitors already in clinical use. Epigenetic drugs increasing expression of miR-15 family members levels or miR762 are anticipated to have therapeutic potential. Importantly, these drugs are generally safe, and could influence NRDs even with small changes in miR levels. No regulators of miR-762 have been published, so whether candidate drugs might alter miR-762 levels in vitro were assessed. Cells were exposed to vehicle, trichostatin A (TSA, an HDAC inhibitor and antifungal agent), and valproate (an epilepsy drug, mood stabilizer, and weaker HDAC inhibitor). TSA increased miR-762 levels 65-fold (FIG. 7). Valproate had a modest but significant effect (FIG. 8). Interestingly, patients with FTD are often prescribed mood stabilizers for behavioral control, and small studies have shown a beneficial effect of valproate. Those studies did not stratify patients by underlying genetic lesion, but it is intriguing to hypothesize that the valproate effect may be greater in patients with C9 expansions where it is boosting miR762 activity depleted by sponging.

### Example 4

### Analysis to Determine Whether In Vitro Models and Human Expression Array Data Indicate Decreased Activity of the miR-15 Family and miR-762 in HD, DM1, and ALS/FTD

In silico analysis and preliminary data suggest possible biologic relevance to the predicted interaction between specific miRs and individual disease-causing expanded nucleotide repeats. However, it remains vital to assess whether this interaction occurs in relevant cells, and whether disruption of miR-specific pathways occurs in patients with disease. DM1 myoblasts show differentiation defects, and it is hypothesized that dystrophic muscle and neurodegeneration in DM1 and FTD-MND may be partially due to impaired differentiation of progenitor cells. Moreover, the miR-15 family has been shown to play a role in NSC differentiation, and miR-762 downregulation of AMD1 is necessary to trigger neural precursor differentiation. Thus, reporter assay experiments will be performed in NSCs. Furthermore, statistical analysis will be used to confirm that the predicted degrees of homology are unlikely to be random, providing strong circumstantial evidence for biologic relevance. To show relevance to human disease, protein and RNA levels of targets of the relevant miRs in human and mouse model tissue will be analyzed. Lastly, available DM1 and HD microarray datasets will be mined to test for altered miR-15 family target genes/pathways.

### Example 5

### Transfecting Human iPSC-Derived NSCs

Human iPSC-Derived NSCs Will be Transfected with a Control

vector or a vector with the HD repeat, then differentiation assayed by immunohistochemistry (IHC) for the stem/progenitor markers CD133 and nestin, the neuronal marker βIII-tubulin, and the astrocytic marker GFAP. Differentiation will be driven by removal of FGF2 from the media. Apoptosis in these NSC-derived neurons will be quantified with a TUNEL assay and a caspase-3/7 activity luciferase assay. These assays will be repeated with a miR-195 sponge vector or control vector, comparing effects on NSC differentiation and neuronal apoptosis with those from the HD repeat vector. In addition, the initial assays will be repeated with the HD repeat vector with co-transfection of a miR-195 expression vector, testing whether the original phenotypic effects on differentiation and apoptosis are ameliorated. Analogous experiments will be performed with an ALS-FTD repeat region vector (with the hexamer repeat from C9ORF72 as an artificial 3′UTR behind GFP), a miR-762 sponge, and a miR-762 expression vector. For in vitro DM1 phenotypic modeling, C2C12 mouse myoblast cells will be used, as well as the miR-195 sponge and miR-195 expression vectors from the HD studies above. Differentiation will be determined by applying differentiation media and quantifying myotube formation.

### Example 6

### In Vitro Models

In the HD and ALS/FTD in vitro models, NSCs expressing repeat region vectors may exhibit impaired neuronal differentiation (though glial differentiation may be intact or increased), and this may improve with forced over-expression of miR-195 and miR-762 respectively. The differentiation defect may be imitated by dedicated sponges for miR-195 and miR-762. NSC-derived neurons may have greater apoptosis with the HD repeat region vectors and the miR-195 sponge, and similar results with the ALS/FTD repeat region vector and miR-762 sponge may be observed. The differentiation defect in DM1 myoblasts should appear in wild-type myoblasts with the DM1 repeat region vector, but be ameliorated by co-transfecting miR-195 expression vector. Positive results will support the NRD miR sponging hypothesis with respect to key phenotypes evident in vitro, as well as yield critical new findings on the biology of these diseases. Regarding the feasibility of these experiments, most of the necessary vectors for these studies have been constructed and most of the rest can be commercially obtained. Alternatively, other neuronal iPSC, such as NSCH14, are available from WiCell, if NSC-H9 cells are found to be difficult to transfect or use in our highthroughput assays. If the sponges do not phenocopy effects from the repeat region vectors, the sponges can be redesigned, or in the case of the HD and DM1 studies focus on miR-15 family members other than miR-195.

### Example 7

### To Test Whether Screens Will Identify Genes and Small Molecules that Elevate Expression of the miR-15 Family and miR-762

One corollary of the NRD miR sponging hypothesis is that agents that elevate expression of the relevant miR for that NRD should overcome the sponging effect and have therapeutic potential. A screen for agents that elevate expression of miR-15 family members for HD and DM1, and also agents that elevate expression of miR-762 for ALS/FTD will be performed. NSC lines stably expressing luciferase reporter plasmids for the relevant miRs or control luciferase plasmids will be developed and used to screen shRNA or small molecule libraries for genes and agents that elevate activity of the relevant miRs. The shRNA library is being included to gain biologic insights into expression of these miRs as well as tractable pathways that might be manipulated with drugs. The compound/drug libraries to be screened include a large unbiased chemical library, three complementary libraries of pharmacologically active agents including FDA-approved drugs for rapid repurposing, and an epigenetic compound library (given the potential of epigenetic drugs to modulate gene/miR expression and our promising preliminary data with an HDAC inhibitor). The resulting leads will be tested individually with the miR activity reporters and the phenotypic assays of Examples 2, 5, and 8. Chemical structural analyses will be employed to quickly obtain available analogs to test in an effort to develop structure/activity relationships to further optimize small-molecule leads. Leads will be filtered with software predicting blood-brain barrier (BBB) penetrability.

### Example 8

### Transducing iPSC-derived NSCs

The iPSC-derived NSCs of Example 7 will be transduced with commercially-available lentiviral vectors with miR activity reporters or control lentiviral vectors. The vectors include copies of miR target regions (seed matches plus extra complementarity) as artificial 3′-UTRs behind the firefly luciferase gene, and also include GFP behind a constitutive promoter. The NSCs will be transfected with lentiviral vectors for miR-195 reporter, miR-762 reporter, or control lacking 3′-UTR sites, then sorted via flow cytometry to select for GFP-expressing cells. Large populations will be generated and frozen to ensure a stable platform over time. The libraries will be screened against the miR-195 NSC reporter line (or the miR-762 NSC reporter line) versus the control NSC reporter line, with “hits” consisting of shRNAs or compounds that disproportionately (z-score=3) decrease luciferase activity in the miR reporter line versus the control line. This should filter out agents that are nonspecifically toxic or that affect luciferase expression/activity. The libraries to be screened include an shRNA library of the “druggable genome,” (Dharmacon On-Target-plus druggable genome library, 7,590 pooled siRNAs), an unbiased library licensed from the Southern Research Institute (25,000 compounds), small molecule libraries (Library of Pharmacologically Active Compounds, LOPAC (1,280 compounds; Sigma); NIH Clinical Collection (˜480 compounds safe but ineffective in Phase II trials), UVA Pharmaceutical Set (2,354 compounds; 7% overlap with LOPAC and 0% overlap with NIH Collection), and an epigenetic compound library (over 75 molecules; Cayman Chemical). Candidate shRNAs or compounds that elevate relevant miR activity will be validated in miR activity assays as in Example 2 and in vitro phenotypic assays of Example 7. The “druggable genome” dataset will be computationally mined using software programs such as Ingenuity Pathway Analysis to identify druggable pathways for pharmacologic intervention. Given the likelihood of numerous hits with the largest effects on miR activity will be prioritized, but also agents with adequate BBB penetrability as predicted by the Pharma Algorithms of the ACD/ADME software (ACD/Labs, Advanced Chemistry Development, Inc.). PubChem, SciFinder, Drug Bank, Zinc and Dotmatics will also be used to seek available analogs in silico to further examine the chemotype altering miR expression and to establish an initial structure-activity relationship for future lead optimization of identified small molecules.

### Example 9

### Data Analysis

Candidates that increase expression of miR-15 family members and miR-762 will be identified, given the preliminary data already suggesting that an HDAC inhibitor may do both. Filtering by BBB penetrability will restrict the number of compounds carried forward for additional testing and enhance their likely utility. As mentioned above, alternative iPSC cells are available, if necessary, and these lines can be re-derived, or initially infect larger quantities of NSCs to generate enough for the screens. Identifying genes that modify expression of the relevant miRs could be of great interest. It is important to note that identifying agents to elevate expression of miR-15 family miRs and miR-762 is likely to be valuable. miR-15 family members have tumor-suppressive function and are under-expressed in certain cancers, so agents that elevate their expression could have therapeutic potential in cancer. Given that miR-762 has been associated with differentiation to neural precursor cells, agents to elevate miR-762 expression may help promote neuronal development and have utility for neurologic diseases.

### Example 10

### Evaluating in Mouse Models of Huntington's Disease and DM1 Whether Modulation of the miR-15 Family and miR-762 can Ameliorate Disease

It is critical to evaluate in mouse models of NRDs whether up-regulation of the miRs being sponged can ameliorate in vivo disease phenotypes. Representative HD and DM1 models (none exist for ALS-FTD) will be used to determine if local delivery of lentiviral miR-195 improves readouts of disease activity. Whether systemic delivery of the HDAC inhibitor vorinostat (SAHA) or of top drug candidates from the previous Examples can also ameliorate disease phenotypes will be tested in these models.

### Example 11

### In Vivo HD Therapeutic Effects

In vivo HD therapeutic effects using the well-established CAG140 knock-in HD mouse model, expressing huntingtin with a 140Q stretch will be evaluated. Homozygous CAG140 mice exhibit hyperactivity at 1 month of age (possible correlating with HD chorea), hypoactivity beginning at 4 months of age, and progressive motor/behavioral deficits. Nuclear and neuropil mutant huntingtin inclusions are detected beginning at 4 months of age, and significant striatal atrophy is observed starting at 24 months of age. The slow progression in this model correlates with adult-onset pathogenesis, and provides a large therapeutic window for testing treatments. To test effects of miR-195 delivery, CAG140 homozygous mice at four weeks of age will be anesthetized and injected in both brain hemispheres with lentivirus encoding miR-195 or control miR. 10⁹ pfu of lentivirus will be injected in 5 μL over 30 minutes to boost delivery volume with a convection-enhanced delivery (CED) process. At 2 months, 4 months, 6 months, and 12 months of age, general motor function and motor learning will be tested alongside controls (wild-type and untreated CAG140 mice) in the activity cage, Morris water maze task, elevated plus maze, and on the Rotarod. In addition, brains of controls and treated CAG140 mice will be examined by IHC for mHTT inclusions (MW8 and mEM48 antibodies) and gliosis (GFAP antibody). As an alternative to lentiviral miR delivery, phenotypic effects of treatment with vorinostat (100 mg/kg daily×7 days every other week) or with leads from previous Examples that elevate miR-195 activity will be tested. Similar testing will be performed in the HSA LR mouse, a widely-used and accepted mouse model of DM1. At day 7 of life, DM1 mice will be anesthetized via xylazine/ketamine, and 5 μl with 10⁸ pfu miR-195 lentivirus/injection (Systems Biosciences) given in the tibialis anterior, soleus, and quadriceps muscles on one leg. Empty lentiviral construct will be given in the contralateral leg. When mice reach maturity (day 60), they will be sacrificed and hind-limb muscles collected. Part of the muscle will be harvested for RNA collection to confirm miR and lentiviral levels. The remainder will be sectioned and dystrophy graded histologically with a scale we helped develop previously, for evidence of protection conferred by the miR. For systemic treatment in the DM1 model, mice will be treated beginning at 2 weeks of age with vorinostat, vehicle only, or lead candidates from previous Examples. Phenotypes will be gauged with established readouts for treatment effect (mortality, muscle strength via grip force meters and treadmill assays, muscle histology, and cardiac conduction health) and compared between treatment and vehicle groups. Muscle tissues and white blood cells will be harvested at selected timepoints to quantify miR-195 and other miR-15 family members by modified real-time PCR across groups.

### Example 12

### Systemic Lentiviral miR Delivery

Local delivery of lentiviral miR-195 or systemic treatment with vorinostat or compounds from previous Examples may ameliorate disease in both models. If miR-195 delivery is ineffective, other miR-15 family miRs miR-15, miR-16, or miR-497; will be tested, while they share substantial homology, there are differences that affect their target profiles. If local miR delivery is difficult in the DM1 mouse model, systemic lentiviral miR delivery will be explored; there is precedent for this in a DM1 mouse model. These studies may serve to test in vivo elevation of miR-15 family miR expression by an HDAC inhibitor and compounds identified in the previous Examples.

### Example 13

### In Silico Analysis Indicates Striking and Specific miR-762 C9ORF72 Nucleotide Repeat Expansion Homology

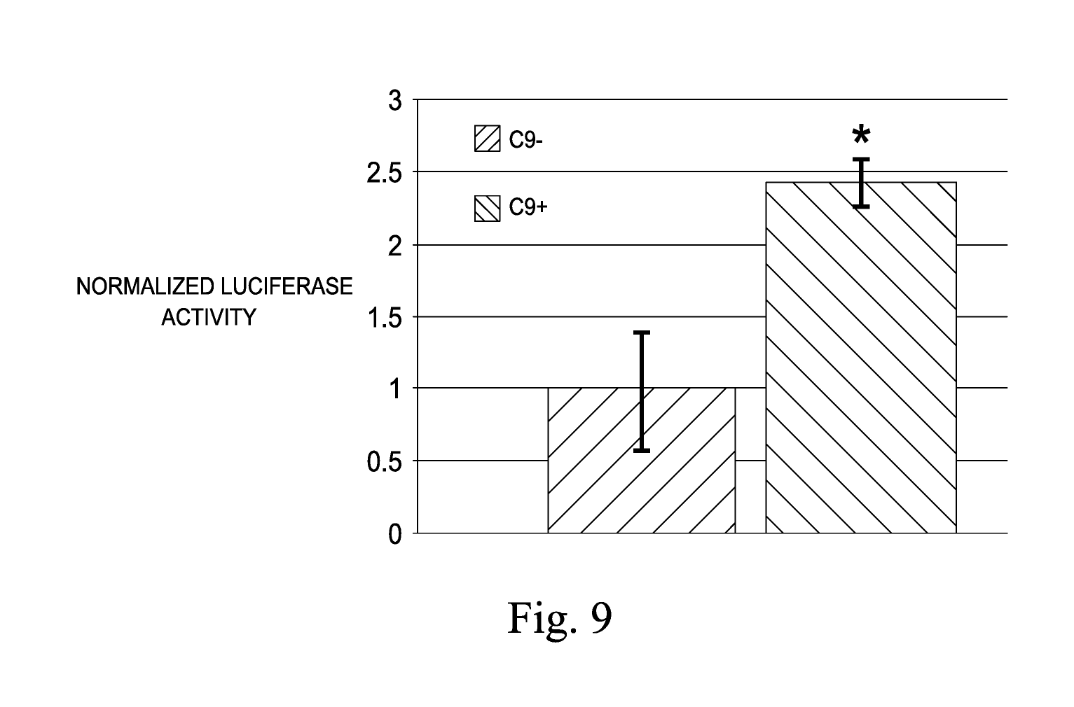

Validated miR target prediction software will be used to assess the described C9ORF72 nucleotide repeat expansion for potential miR homology. Both sense and antisense repeat transcripts will be used as inputs, based on reports suggesting that antisense transcripts are produced in abundance in these patients. For C9ORF72, a very striking match between the antisense repeat and miR-762 was identified (Table 1). While little is known about miR-762, it has been linked to neural stem cell (NSC) differentiation. Based on the strength of the predicted homology and the known biology, direct miR-762 binding to the inappropriately expanded repeats may serve to “sponge” the miR. This sequestration would then render miR-762 unable to bind its normal downstream targets, leading to cellular dysregulation and disease. To test whether patients carrying the C9 expansion has altered cellular miR-762 function, a miR-762 reporter plasmid will be introduced into patient cell lines. miR-762 activity is diminished in naïve cells containing transfected repeats (FIG. 7) and in C9+ patient cells despite no change in miR-762 levels (FIG. 9), consistent with the sponging hypothesis.

### Example 14

### NETO1 is Upregulated in C9 Expansion Carriers

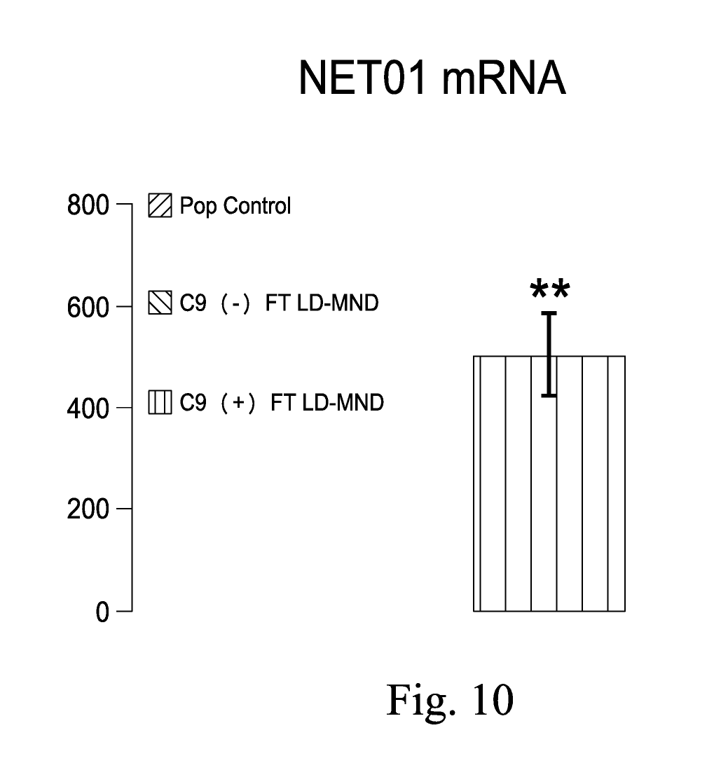

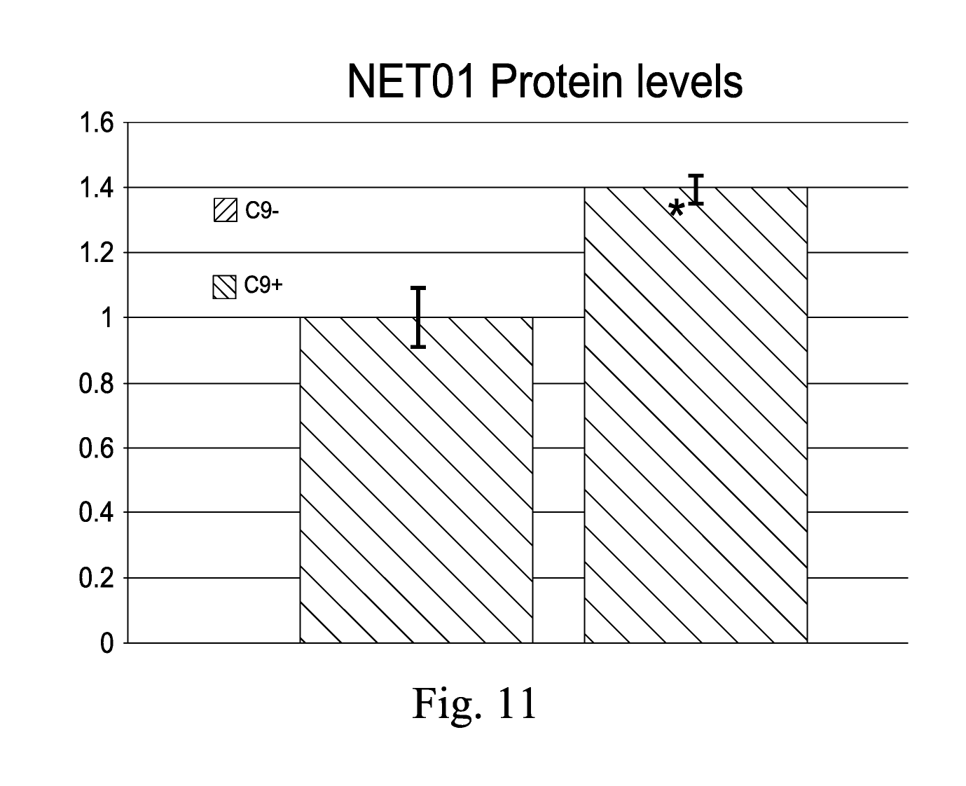

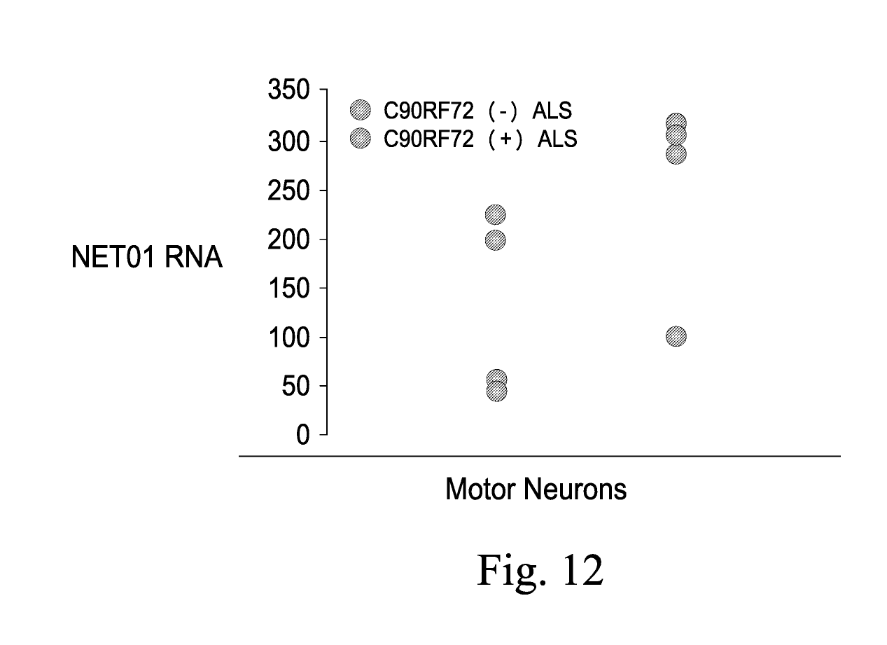

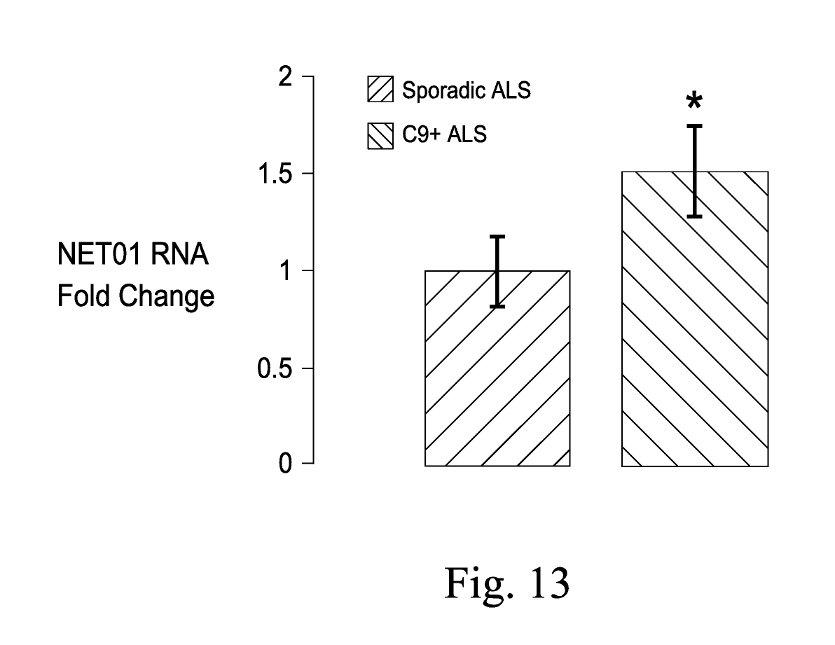

Based on the theory that miR-762 targets may be dysregulated in C9+ patients, mRNA patterns in C9+ versus C9-FTLD-MND patients (n=3 each) was analyzed via microarray comparison. One of the most differentially regulated targets identified was NETO1 (Neuropilin tolloid-like1) (FIG. 10). NETO1 mRNA upregulation was observed in patient lymphocytes and demonstrated commensurate (though less dramatic) protein upregulation as well (FIG. 11). Using publically available GEO Datasets, a trend towards upregulation of NETO1 in young C9+ iPSNs was evaluated as well (FIG. 12). NETO1 is a predominantly CNS-expressed protein with 2 CUB-domains and 1 low density lipoprotein A motif which is known to localize to the PSD. Netol has been linked to function of all three ionotropic glutamate receptors; it modulates gating properties and synaptic localization of KARs and affects subunit balance and trafficking of NMDARs. The NETO1 homolog has been defined as auxiliary subunit of AMPARs in C. elegans as well. NETO1's role in glutamate signaling and upregulation in C9+ patient cells is intriguing in light of known glutamate dysfunction in C9 expansion carriers. Importantly, in autopsy frontal cortex samples from C9+ and C9− patients, NETO1 was similarly upregulated, confirming the in vivo relevance of the finding (FIG. 13). NETO1's role in glutamate signaling and upregulation in C9+ patient cells is intriguing in light of known glutamate dysfunction in C9 expansion carriers.

### Example 15

### NETO1 Expression is Independently Affected by the Presence of C9 Repeats and by miR-762 Levels

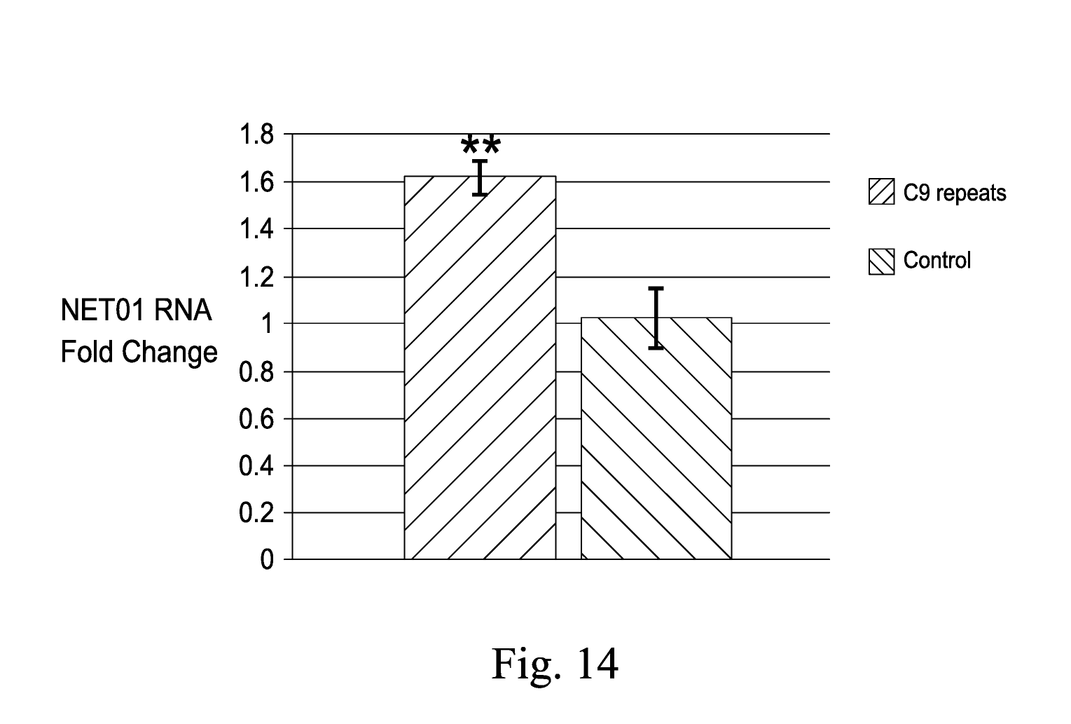

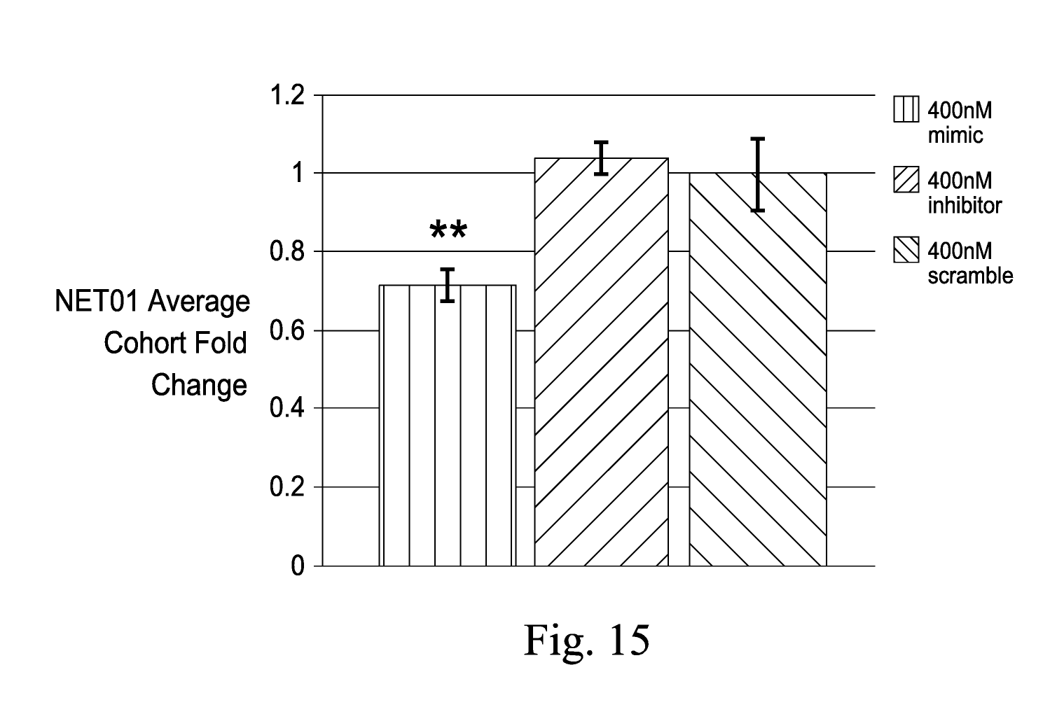

De novo introduction of the expanded repeat into naïve wild-type cells to induce NETO1 expression will be evaluated. Transfection of a construct containing 12 C₄G₂ repeats may be sufficient to significantly induce NETO1 mRNA upregulation (FIG. 14). NETO1 is not a predicted target of miR-762 by traditional in silico analysis (which relies on 3′ UTR analysis). However, a (C₄G₂)₃ site 149 base pairs upstream of the NETO1 5′ UTR was identified that appeared significant both for its perfect replication of the C9 antisense expansion and its predicted tight pairing to miR-762 (RegRNA database: http://regrna.mbc.nctu.edu.tw/html/prediction.html). miR binding and regulation of transcripts in the 5′ region is poorly understood, but is known to occur. miR-762 may be able to bind and downregulate NETO1 transcript levels. To assess this, non-diseased astrocytes were transfected with 400 nM miR-762 mimic, miR-762 inhibitor, or scrambled miR. Introduction of miR-762 resulted in a reproducible and significant downregulation of NETO1 mRNA (FIG. 15). If NETO1 regulation is involved in FTLD-MND glutamate excitotoxicity, then targeting miR-762 regulation of the transcript could represent a possible therapeutic angle.

### Example 16

### Determine if NETO1 is Upregulated in FTLD-MND Patient Tissues

Frontal cortex and spinal cord samples will be obtained. The sample cohort will contain 8 C9+ and 8 sporadic disease samples. mRNA and protein from all samples will be collected and analyzed for NETO1 expression levels. Though the original analysis identified NETO1 as upregulated in C9 familial disease when compared to sporadic cases, other genetic fALS will be analyzed to explore NETO1 upregulation. Sample size may be increased to analyze other familial cases.

### Example 17

### NETO1 Level Changes

Using published Geo Datasets, a trend towards NETO1 upregulation in C9+ iPSNS at 3-4 weeks growth was discovered. NETO1 level changes with neuronal age will be investigated using the described iPSNs. NSCs will be differentiated into neuronal cultures using standard techniques commonly employed. Briefly, NSCs will be expanded on matrigel in NPC media (DMEM/F12+ Glutamax, supplemented with 1% N2, 2% B27 without Vit A, 20 μg/mL FGF2, and 1 mg/ml Laminin). When passage 4 at 70% confluence is reached, the cells will be plated onto commercial Poly-D Lysine laminin coated coverslips (Neuvitro Cat # CG-25-Laminin) and co-cultured with human cerebellar astrocytes. At 70% confluence on coverslips, media will be changed to DMEM/F12+ Glutamax supplemented with 1% N₂, 2% B27 with Vit A, BDNF 20 μg/mL, GDNF 20 μg/mL, Cyclic AMP 500 nM, Ascorbic acid 200 μM, 1% Pen-Strep) to promote neuronal differentiation. Neurons will be grown to 2, 4, and 6, and 8 weeks. Protein and mRNA will be collected from each culture and analyzed initially for NETO1 expression. NETO2, Grik1/2/3/4/5 (Kainate), Grin1/2a/2b/2c/2d (NMDA) and Gria1/2/3/4 (AMPA) subunit mRNA expression levels will be examined based on the knowledge that other familial FTLD-MND models and cell lines display alterations in these components as neurons age.

### Example 18

### NETO1 Overexpression

NETO1 overexpression in mature adult wild-type iPSNs inducing glutamate excitotoxicity will be determined. An inducible vector for NETO1 overexpression will be constructed using commercially available expression ready hNETO1 constructs and transfected into two independent neurotypic NSC lines. Following differentiation, expression will be induced in a subset of the samples, and the induced and uninduced iPSNs will be subjected to a standard glutamate toxicity protocol: Briefly, the cultures will be exposed to varying concentrations (1, 3, 10, 30, and 100 μM) of glutamate and cell survival will be observed with propidium iodine uptake. To determine if neuronal cell death is glutamate receptor mediated, we will treat cells with glutamate receptor and calcium channel inhibitors (MK-801, 10 μM; CNQX, 10 μM; nimodipine, 2 μM). NETO1 expression will be quantitated in both induced and uninduced iPSNs to assess level of expression.

### Example 19

### Assessing if Downregulation of NETO1 Abrogates C9 iPSN Glutamate Excitotoxicity

The prepared C9 iPSNs will be assessed to determine sensitivity to glutamate. To assess if NETO1 participates in the observed excitotoxicty, NETO1 will be knocked down using siRNA technology. Two independent NETO specific siRNAs will be obtained from commercial sources (Santa Cruz Biotechnology and Life technologies). Pilot knockdowns will be done using 10 μM and 30 μM siRNA with time curves to measure mRNA and protein expression levels. The most effective siRNA will be used in additional experiments. C9+ iPSNs will be transfected with NETO1 siRNA using Lipofectamine RNAiMAX (Lifetechnologies). The age of neurons chosen will depend on timing of NETO1 upregulation as determined in the previous Examples. Appropriate positive (Hypoxanthine-guanine phosphoribosyltransferase) and negative (scrambled) siRNA controls will be employed. Standard glutamate toxicity assays will be used to assess excitoxicity in siRNA treated iPSNs compared to controls.

### Example 20

### Identifying C9 Repeat Expansions

A (CCCCGG)₃ site upstream of the NETO1 5′UTR that is identical to the antisense C9 repeat expansion sequence was identified. This occurs relatively rarely in the genome, and is intriguing given the proposed mechanisms associated with toxic RNA, namely sequestration of a regulatory molecule (miR or protein) by the C9 expansion leading to altered binding elsewhere. Given alterations in NETO1 expression in C9+ expansion carriers, this sequence may be important for regulation of NETO1 expression and key to NETO1 overexpression in the disease state.

### Example 21

### Assessing if Mutation of (C₄G₂)₃ Abolishes C9-Repeat Dependent NETO1 Upregulation

Introduction of vector based C9 repeats can cause an upregulation in endogenous NETO1 in a wild-type astrocyte line. Using standard cloning techniques, the region upstream of the NETO1 start site with the intact (C₄G₂)₃ or scrambled sequence without miR-762 predicted homology will be cloned into a luciferase expression vector. Once the integrity of the vector is confirmed, each vector will be co-transfected with the C9 repeat plasmid (or empty control vector) into immortalized astrocytes and upregulation of the reporter in the presence of antisense repeats will be determined. The experiment with the reporter plasmid will be repeated with the mutated upstream site to assess if that abolishes reporter upregulation. This allows for a direct assessment of the role of the (C₄G₂)₃ site on Netol expression.

### Example 22

### Assessing if miR-762 Effect on Netol Requires the (C₄G₂)₃ 18-mer Site

Similar to Example 15, expression vectors will be used to examine the role of the 18-mer on miR-762 NETO1 dynamics. Each expression plasmid (containing CG-rich 18mer or scramble) will be co-transfected with 400 nM miR-762 mimic, miR-762 inhibitor, or scrambled miR sequence into immortalized astrocytes. Given the high predicted homology between miR-762 and the 18mer, a downregulation of the reporter in the presence of the mimic and no effect of the mimic on expression of luciferase in the system utilizing the mutated 18-mer site may be observed.

### Example 23

### Identifying Proteins Capable of being Sequestered by the (C₄G₂)n Repeat Sequence

The antisense RNA is produced in as high abundance as the sense strand, and is present in affected cell types in the cortex and spinal cord. Therefore, the antisense strand may also have RNA-protein binding and sequestration potential. Computational prediction tools will be used to identify one potential binding partner RBMX, a known RNA binding protein shown to alter SOD1 mechanics and TDP-43 biology when knocked down. To identify possible binding partners, the creation of a Cy5-labelled (C₄G₂)₇ repeat oligo (IDT) will be commissioned. A GC-rich scrambled Cy5 labeled oligo to control for non-specific binding (5′Cy5-CCCGCCCCGCCGCGCCCCCGCCCCGCCGCGCCCCCGCCCC (SEQ ID NO: 11) will be prepared. Proteome array analysis will be completed with 250 nM input oligo), as described, using a HuPro Human Proteome Microarray. Three arrays will be hybridized per oligo in parallel to increase data reliability. The data will be processed and using standard array statistical tools. Cellular colocalization of putative binding partners will be confirmed with antisense foci using published RNA-FISH immunofluorescence protocols. Furthermore, the in silico hit, RBMX, will be directly tested with co-localization assays. Additional RNA co-immunoprecipitation with antisense RNA will allow for confirmation of in vitro co-localization.

### Example 24

### 3D Modeling of C9 Neural Cultures

The development of patient based iPSCs from FTLD-MND affected individuals has offered a powerful new tool for the study of disease pathogenesis. iPSNs in 2D have proven useful for recreating some of the pathogenic features of disease in culture. However, the two dimensional neural cultures have significant limitations. Neurons in 2D do not live as long as those grown in 3D, limiting their utility when studying diseases of aging and neurodegeneration where time equates with accumulating pathology. Furthermore, complex interactions between neurons and glia are more accurately represented in 3D. The local concentrations of secreted proteins (important in neurodegeneration) are higher in a 3D matrix than when diffused in media on a 2D platform, and precedence exists for superiority of 3D modeling in neurodegenerative diseases (PD, AD references) like Parkinson's disease and Alzheimer's disease. Based on these observations, a 3D modeling of C9 neural cultures will be created. Such a culture can be manipulated to bias neuronal/glial ratios (and genetic composition), interrogated in novel ways to study synaptic glutamate regulation, and designed to assess effects of drugs targeted known pathogenic pathways.

### Example 25

### Develop a 3D Scaffolded Matrix of C9+ and C9-FTLD-MND Mixed Neuronal/Glial Cultures

Four C9+ FTLD-MND, two C9-FTLD-MND, and three neurotypic control NSC lines have been derived from iPSCs. One line from each genotype will be used to develop the assay. First, Alvetex® Scaffold circles will be treated with 70% ethanol followed by two PBS washes, followed by addition of 500 μl of poly-D lysine per well and appropriate incubation. Neural rosettes will be collected via Stemdiff selection reagent (Stem Cell Technologies) and cell suspensions will be seeded directly on the wet poly-L-lysine coated Alvetex® Scaffold membrane at a target concentration of 2×10⁵ cells/cm². After 4-5 days, the media will be switched to standard differentiation media (Example 17). Immunohistochemistry will be performed with the following cellular markers: β-tubulin III (immature neuronal), GFAP (astrocyte), V-GLUT1 (glutamatergic cell type), MAP2 (mature neurons) and O4 (oligodendrocyte) to ensure adequate neuronal/glial co-culture. Once the cultures are created, cell morphology, cell viability, and neurite outgrowth will be followed over the lifespan of the culture to determine basic culture characteristics.

### Example 26

### Determine if 3D C9+ Cultures Develop Key Pathologic Features of C9+ FTLD-MND

Three of the key pathologic features of C9 disease are:
1. Intranuclear TDP-43+ and cytoplasmic p62+ (TDP-43-) inclusions;
2. Nuclear and cytoplasmic foci of both sense and antisense C9+ mRNA;
3. Accumulation of cytoplasmic dipeptide products of RAN translation.

To validate that the 3D model recapitulates pathology known to occur in patients, detailed protocols previously published for identifying and localizing these three key pathological features of C9ORF72 FTLD-MND will be followed. This is similar to the approach taken for validation of published 2D iPSN models. Furthermore, recapitulation of glutamate excitotoxicity will be confirmed using assays.

### Example 27

### Assess if miR-762 Expression Rescues Pathologic Features of C9+ Expansion

The sponging model predicts that raising levels of free miR-762 affects rescue of certain pathologic disease features associated with the repeat expansion, particularly glutamate excitotoxicty. A lentiviral doxycycline-inducible miR-762 expression construct will be transfected into C9+ NSCs prior to seeding onto the 3D scaffolding. Once seeded and expanded, differentiation into mixed cultures will proceed as described in Example 17. Once mature cultures are obtained (week 2) miR-762 expression will be induced by addition of doxycycline to the media (controls will we remain uninduced). After 3 days of miR-762 expression, glutamate toxicity will be assessed.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

1. A method of treating a nucleotide repeat disorder (NRD) in a patient comprising
detecting the presence of an NRD repeat in a biological sample recovered from said patient; and
treating those patients having a said NDR repeat by administering an effective amount of a therapeutic that enhances the activity of the miRNA that corresponds to the detected NRD repeat.

2. The method of claim 1, wherein the therapeutic is an HDAC inhibitor.

3. The method of claim 2, wherein the HDAC inhibitor is selected from the group consisting of trichostatin A, valproate and vorinostat.

4. The method of claim 2, wherein the HDAC inhibitor is valproate or trichostatin A.

5. The method of claim 1 wherein the therapeutic is a composition comprising
one, two or three compounds selected from the group consisting of trichostatin A, valproate and vorinostat; and
a pharmaceutically acceptable carrier.

6. The method of claim 1, wherein the biological sample is a blood sample.

7. The method of claim 1, wherein the step of detecting comprises
contacting nucleic acids recovered from a biological sample with a reagent that specifically binds to an NRD repeat.

8. The method of claim 7 wherein the reagent is a nucleic acid sequence that binds to a sequence, or a corresponding compliment thereof, selected from the group consisting of SEQ ID NO: 2, SEQ ID NO: 4, SEQ ID NO: 6, SEQ ID NO: 8 and SEQ ID NO: 10.

9. The method of claim 7 wherein the therapeutic enhances the activity of an miRNA selected from the group consisting of SEQ ID NO: 1, SEQ ID NO: 3, SEQ ID NO: 5, SEQ ID NO: 7, and SEQ ID NO: 9.

10. A method of treating ALS in a patient comprising
screening for the presence of a C9 expansion sequence comprising SEQ ID NO: 6 in a biological sample recovered from said patient; and
treating patients having said C9 expansion with a therapeutic agent that
a) enhances activity of miR-762, or
b) decreases activity of NETO1.

11. The method of claim 10, wherein the therapeutic agent comprises an HDAC inhibitor.

12. The method of claim 11, wherein the HDAC inhibitor is selected from the group consisting of trichostatin A, valproate and vorinostat.

13. The method of claim 11, wherein the HDAC inhibitor is valproate or trichostatin A.

14. The method of claim 10 wherein said treating step comprises administering a composition comprising
one, two or three compounds selected from the group consisting of trichostatin A, valproate and vorinostat; and
a pharmaceutically acceptable carrier.

15. The method of claim 10, wherein the step of enhancing activity of miR-762 includes increasing the effective concentration of miR-762.

16. The method of claim 10, wherein the step of increasing the concentration of miR-762 comprises administering a lentivirus comprising miR-762.

17. The method of claim 10, wherein the step of decreasing activity of NETO1 includes enhancing the activity of miR-762.

18. A kit comprising
a) an oligonucleotide that specifically binds to a nucleic acid, or a corresponding compliment thereof, selected from the group consisting of SEQ ID NO: 2, SEQ ID NO: 4, SEQ ID NO: 6, SEQ ID NO: 8 and SEQ ID NO: 10; and
reagents for detecting the binding of said oligonucleotide to its target sequence.

19. The kit of claim 18 further comprising an HDAC inhibitor selected from the group consisting of trichostatin A, valproate and vorinostat.
